SEQUENCE LISTING (18 sequences)

SEQ ID NO: 1 - DNA, 726 bp, Cassava vein mosaic virus
  source 1..726: mol_type = genomic DNA
tttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgatttccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttg

SEQ ID NO: 2 - DNA, 583 bp, Blueberry red ringspot virus
  source 1..583: mol_type = genomic DNA
tttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgatttcatccaggacactactgtccaatattgtccattcattcagcagggatgataagaagtcagtatcaagtgaagacgaagaaaagatcacccatggaagtggaagaataaaacgtcttcatccatcactatcaagacgtagacatcacaatgtaaagctacgggctaatattagatggcttaagaatctacgccaatgtaaagtagattccccttatcttttatttttaaagtttttaagtccggagttgagttccgtcctgtgggaattcctctatataaggacgacctatcctatgtttgtagtctatttttacaacaattaccaacaacaacaaacaacaaacaacattacaattactatttacaattaca

SEQ ID NO: 3 - DNA, 592 bp, synthetic construct
  source 1..592: mol_type = other DNA
tttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgattttacagagtttttacttcggacagtcaaaaatgagtttaacttctcagccgaggtaaaacaagaaatatgcttacgtctacagagggatttctctgaagatcatgtttgccagctatgcgaacaatcatcgggagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcatccgctttgcctataaataagttttagtttgtattgatcgacacgatcgagaagacacggccat

SEQ ID NO: 4 - DNA, 371 bp, Cauliflower mosaic virus
  source 1..371: mol_type = genomic DNA
ctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggct

SEQ ID NO: 5 - DNA, 211 bp, Figwort mosaic virus
  source 1..211: mol_type = genomic DNA
tttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgattt

SEQ ID NO: 6 - DNA, 443 bp, Cassava vein mosaic virus
  source 1..443: mol_type = genomic DNA
ccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaac

SEQ ID NO: 7 - DNA, 8159 bp, Cassava vein mosaic virus
  source 1..8159: mol_type = genomic DNA
tggtatcagagcttagtttaaaaataataatggatagtaaagattttacacaacttaatctagaagaacatagttataaagttaatagagaaaaattacctatagactcatatattcagtatggaggatttacttatgcaaattttacaccatatattatacatggagatgatggatttggagaacataaacaactgaattggactaataaattattatggaataaattaggtaaattaaatatcaaagatacacaaattctaatgcagaataatattagtgaagaacaacataatgaattaataagcttagaagcacaaaaaatagctagagaaaatttagctgatagaattaattatttacaaaatattaatacttcaatagattttaagttatggaaaatgaataaagaaaatttagaaaggcaagaattattattaagacaaataaatgaattaaaagaagaaataaaatcattaaaaaatataccaagtacagtagcaataataccaactaatacttatactattaatatgattaggacagaaacagaagattggaaatattttaaatatattgaaaaagaattagttcagaataaaactgaagcaatagcaaaaatattagataatagttatataataaatgataatttaggattattatatgaaagatatgaagaaattaatcccacacctaaaccatataaaagaccagaaactatatttgatacacctcaatatgcaaaatatattaggaatcaaaaaagacaagaagagtatgaaaaacaacaagaattaaaaaaggaaaatgaaaataaagaataccaagaatttttagaatggaaagaaaaacaacaaaaagataaaggtaaaggaatacaaactgtatacccgacattaataataccagatataaaacctgaaaaacaaaagaaagaagatatgatgttagaaatgattaaaaatttacaaaatgaattagaacaattaaaaattcaaagacataaagaacacgaaaaacaagcagagttaactaaaatacagatgttagaagaggaattagaagaagaattagatcctgataatctagaaaaagaagttttaaataacattcaaaatatacaaatcagtagtgatataagtgaatcatcagagataaatgaaatttcagataacgaaacagaacaaatttcaggaagtgatagtgattataataatgaacaaattaatgttaagatagaaggagaagaatatgaatacaaagacaattatagatattataaaccacaaccaccatattacaaaaaagatataagaagagaaaggcaatataaaggtcaaagttcacaaagagcagattacataaaaaacagaagagaacaatttgaatctacatatcaagcaaatatgaatacaaccataaacgatagtggagaaattcttaatttagattgcacaacaccagaggaagcagaagatagaatacaaaaatggactcaatctatgtcaatagcattagttaaacaacaattatctaatgaacaagcaaaacaattcataagaagaacttttataggaaatgtaaaagaatggtataaaaatttaacaaatgaagctaaacaaaaattagaagggaatgcaccattactaagtttaactcatatggaattaggacttagagcagaatttggaaaattaggaatagaatcagatgtagaaaaacatgaaaagaaaacatctattgcaagacataaaatattacaattacaaatatgtagtatggatcatcaaaacttaaatgcatatttatgtgaatttcaagaatattattatagtgcaaattatacagaagcagaatcagaaaatatattaaatatgttttatagtaaactcccagaaccatggggacaacaagtattaaatggatatttatcagaaataaaaggtaaaaatttactagatagtattggagctcgaatgacatatttacaagaatttatatcagataaatgtaaagaaaactggacacaaaaacaagctagaaaaatacaattatcaaagaatctagattgtagctattatgaagtaggaaaatatggatgtaaacaaataagacctcataagagaaaaagatattataaaaaatatattcctataaaaaggaaatattttaataagaaaagatacaaaaaatattatagacctaaaaaattcttaaaaaggaaaaatccgcataaagcatgtaaatgttataattgtggagaagaaggacacataagtccaaattgtaaaaaaccaaaaaagaaaactagaattaataatttagaagctttagaatttaaaaatactgagatggagaatctagaatttgaaacaaataaaaatgatataatatgggtagaagaaatagaagttatacaacctttacattatgaagaagaagaaaaatacaaaggaaattactcagatagaatattacaaaatccttattatataaattcaataagtattgaagaattagataatttagattgggaatttgaatatcaagaagatattgaagatgatttagaataccaaaattttgtatatcaagaatcaaatgataattggtatagtgatcaagaaaattggtatagtgatgaacaatatttaggaatatatatgtttataggagaaacatcaggagaaaataatcaagacaatatggaaggaataataaaagaatataataaaacagaaccagaaaaaataaataaaataatatttacctcagaaaaatttaaacaaattatggaaaatgatttaaatatgacgaaagataaaatatttcataataataaacttaaaaaattatttggaaagaaagaaatagaatactatatagttacagatatcgaacatcctattgatgtaaaatatgtacaaaatcaagataaaataataaacctaccattatataatcaagaaatttttgaaaatgaaatacaaaaaatacctgacaaggatcaaaataaaataagaaatatacatttagcagcagtagaaattgtagtaaaagcatattttagagaaggaatagacacaccatttgaaattatattatgcgatgatagaataacatatccacaagagggaagtttagtagaagttttaataggaaatttaatatatcagaaagtaaaatttacaaaaataattaattattctataagcatagaagataaaaatttagataaaagtttagtaatgtattggaacttagaaggaattaaaatgataaaagatagcaaaatatttagtataagattaagaaatctttatgttttaagtaataaacatatagtgaaaaataaaaaacaatataatggaaatataattattgaacctatatttcaagatgtaatacaaaataataatagaaattatatagaatatggtaaaccaggaaaatttgatagaacaaaattaaaatcatatagtagaagatttaatgaaccgttaagacttgatgatagaacaaatatccagagagaaaaggaccaaatcgaaaaggcagaccataatttagaattacaaaaagaactaaataatttaaattattattctcaacaaggacaaagttctaatgttttagatataccaaaaatattaaagatagagaacactaagaataattataagcaatttcatattattggaaaaattactgaaggaagattaaataaattttatccgatattaatagatacaggagcagcagattcatatataagttcaaagatcttagaagatgagaaattagtttcaaataaattatctaaagtagttacatcatataacgcagataatgaaaaacatatttatgacagaaacacagaagtaataattgaattaatagataaaaataatgaaaaatataaaatcaattttatcggattagtagatcaattaagacttttagaaggaggaaaagctgaaatattattaggaatgaacatattacaaaatttaaaaccctattgtataacagatgattacctagaaattaatttaggctttagatgtataaaaattaatagaataaagaaagatattttcgaaattagagaagatttacaacaaatgaatgtccttaatgaatagtacaaatccagaatttatagaatattatcatacttcatggaaaccacataaaatagaattagttgataatatatatcattcatatggatattatgtgtatacaagaagtgtaataaaaagatttaataaacatttaattaaaactacatataaaagaatatttagtcatccagaaaatatagtattgcattttagatgaataagattacctatatgacaatcaaaatatcaatacctaaatatatgtcaagaatttatcatggattgtttgatactggagcaaatatatgtatttgtaaaaagaaagttttaccagatgaattatggcataagacagaaaatttagtactaagaggatttaatgatgaaaagcatgttgctgaatatagagcagataatataacaataatgatagcaaaagaaaaatttataatcccatatatatatgctatggatgaaatgtcaccagatattattattggagcaactttttataataaatatagtcccatagaactagacataggaaaaggaattataaaatttacaaaaaataatgaaaaatacccaaattatttagtaaaatatcctaagaagagaaaattagtaccatggactaaaggaaacccgagtgttactgaaacaatggaaaatataggaataaatcagattgaaagtagaaatcctatagaagaagaaataaatcaaatattaggaacagatatatatggcgaaaatccattagaaaaatgggaaaaacataaaacattagcaaaaatagaattaaaaaatgaaacagataatatttataaaccaccaatgttatatcaagaaacagatttaccagaatttaaaatgcatatagaagaaatgattaaagaaggttttatagaagaaaaaactaattttgaagataaaaaatattctagcccagcttttatagtaaataaacattcagaacaaaaaagaggaaaaactagaatggtaattgattataaagatttaaataaaaaagctaaagtagtaaaatatcctatcccaaataaagatacattaatacatagaagtatacaggcaagatactatagtaaatttgattgtaaatcaggattttatcatataaagttagaagaagatagtaaaaaatatacagcttttacagttccgcagggatattatcagtggaaagtattaccatttggatatcataactcaccaagtattttccaacaatttatggatagaatatttagaccatattatgattttattattgtgtatattgatgatatattagtcttttctaagactattgaagagcataaaatacatattgcaaaatttagagatataacacttgcaaatggattaattattagtaagaaaaaaacagaattatgtaaagaaaaaatagatttccttggagtccaaatagaacaaggaggtatagaattacagcctcatattattaataaaatattagaaaaacatacgaaaataaaaaataaaactgaattacaaagtatattaggattattaaaccagattagacattttataccacatttagcacagattttattacctatacagaaaaaattaaaaataaaagatgaagagatttggacttggactaaagaagatgaagaaaaaataaaacttatacaagattatagtaaaaatttagtaataaaaatgaaatatcctattaataaagaagatatgaattggattattgaagtagacgcaagtaataatgcttatggtagttgtttaaaatataaaccgaaaaactcaaaaatagaatatttatgtagatataactcaggaacttttaaagaaaatgagcaaaaatatgatattaatagaaaagaactgattgcagtataccaaggattgcagagttattctttatttacatgtgaaggaaataaattagttagaacagataattcacaagtatattattggataaaaaatgatactaataaaaaatcaattgaatttagaaatataaaatatttattagctaaaattgcagtctataatttcgaaattcaactaatagatggaaaaacaaatattattgctgattatttgagcaggtacaattcatctgatactgatggaagatatgatgaagcaaatacttgagaaactcaatacaattgaaaaaaatatttctgaaacaaatatacgaattgaaaaaatagaaaaagaacaagagcttaaaagaaaagtagagctctatggaaaagaaccagagaaaaaattacataaagaaaatatagaaaaattaagctcttctattgaagacaaaatcatacaaaatattgataagaaattaaagaaaattgaaaatgtcgaagaacaatatcagtggaaaaatatagtcaaaataaataaaccactttctgtgggagaaaaatatatggagaatttcaaaaagatacttgtttatcttggagaaaaacatcctaaattggaagaattatattctttgacagattacaacaaattagtagcagatatttatactgatagaaatttagtaatttcagcatataattatggattacttcaagtgctttatattgagcatccgtctcaattagagttatttgatgaaaatataaaacttgcttatatgaaatttagaaatgttaccaaggcacaacttatatatatgcgaatttacagtgcaatggctgaaccctatgataaaggggtgattccaaaaattgaaataattaaatttggaattacttattcaaaacttaaatatgatgaagtatatgaacatcaaccaatagaaaaacttgatttgaaaaaatttatatcacaaaaaagagcattagggatacttgttatccaaaaagaaatagaaaacttgaatggaaacgtatggttgtactccaacctagatggaagacttatattatcaaatcacaacaaagcagaaaatgtagaagtcaaaaatatattatcagattggtcaaaaaaattatccattccagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaatgtttttagtttttataataatatgtttatgtttgttttaataatgagtgagtagtttcttaattcttctaattaatatgaaataattatgctgtctagaaacaagtttaaatctgtttgatattcccagaagctatgaactgttacgatctagataatctttcaattatccccatgatatcctgcgtgtgatattactgagccctttatagaccatgtgtgtcgtttgtatgggcatgaacatctgtgtatgttagggagaaagagagattgaatgatcattgaaagttttaatgaagggaatcaaatgtttatctattacttgttttctatcagacaatagcataattagagtagtattaattaggagaatgttaaaaatattattgttttcactcttagttttaattatcctttgttttatcgatcctatattattttactttatttgtttataattccgctttcttttcacccctt

SEQ ID NO: 8 - DNA, 72 bp, Cassava vein mosaic virus
  source 1..72: mol_type = genomic DNA
aagaaaaaatttggtgtaagctattttctttgaagtactgaggatacaacttcagagaaatttgtaagtttg

SEQ ID NO: 9 - DNA, 800 bp, Cassava vein mosaic virus
  source 1..800: mol_type = genomic DNA; note = each n = any
nnnnnnnnnnnnntcttctcctttannnncctcccggggatcctctagacaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctacgtattcttcttcgtattctggcagtataggatcttgtatctgtacattcttcatttttgaacataggttgcatatgtgccgcatattgatctgcttcttgctgagcttacataatacttccatagtttttcccgtaaacattggattcttgatgctacatcttggataattaccttctggaaatcatacgtcagcgcttacgtttttaagcattaaaatctctcgtgaggcaatctgctaaaacgttttttacaccttcaagatgttcgacatcaaattgatacttgctgaaccaattttgccaacggactaatcgtccttgtttactatcacctttaagattaattctaaggaaataagtaaaatttttgttatcagttcttactgtaaaaagcttcggccggccgttccatccc

SEQ ID NO: 10 - DNA, 305 bp, Blueberry red ringspot virus
  source 1..305: mol_type = genomic DNA
catccaggacactactgtccaatattgtccattcattcagcagggatgataagaagtcagtatcaagtgaagacgaagaaaagatcacccatggaagtggaagaataaaacgtcttcatccatcactatcaagacgtagacatcacaatgtaaagctacgggctaatattagatggcttaagaatctacgccaatgtaaagtagattccccttatcttttatttttaaagtttttaagtccggagttgagttccgtcctgtgggaattcctctatataaggacgacctatcctatgtttgtagtc

SEQ ID NO: 11 - DNA, 67 bp, Tobacco mosaic virus
  source 1..67: mol_type = genomic DNA
tatttttacaacaattaccaacaacaacaaacaacaaacaacattacaattactatttacaattaca

SEQ ID NO: 12 - DNA, 20 bp, synthetic construct
  source 1..20: mol_type = other DNA
gccccgtaatgcagaagaag

SEQ ID NO: 13 - DNA, 20 bp, synthetic construct
  source 1..20: mol_type = other DNA
tcttgacctcagcgtcgtag

SEQ ID NO: 14 - DNA, 24 bp, synthetic construct
  source 1..24: mol_type = other DNA
tccaatccgagtcgctgtcattgt

SEQ ID NO: 15 - DNA, 24 bp, synthetic construct
  source 1..24: mol_type = other DNA
tgatgagcctctctggtttgacct

SEQ ID NO: 16 - DNA, 301 bp, Figwort mosaic virus
  source 1..301: mol_type = genomic DNA
tttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgatttcaaaaaacgcagctataaaagaagccctccagcttcaaagttttcatcaacacaaattctaaaaacaaaattttttagagagggggagtg

SEQ ID NO: 17 - DNA, 12830 bp, Figwort mosaic virus
  source 1..12830: mol_type = genomic DNA
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagttttacggccggccgaagctttttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgatttccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtctagacatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcagcatggacgagctgtacaagtaagagctcaccggtcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattgcggccggccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgactagtggtacaattctcaacacaacatatacaaaacaaacgaatctcaagcaatcaagcattctacttctattgcagcaatttaaatcatttcttttaaagcaaaagcaattttctgaaaattttcaccatttacgaacgatagcctaggatgattgaacaggatggtcttcatgctggatctccagctgcatgggttgaacgactctttgggtatgactgggcacaacagaccataggttgctctgatgctgcagttttcaggcttagtgcacaagggagacctgttctcttcgtcaagactgacttgtccggagctttgaatgagcttcaggatgaggctgccagactttcatggcttgctacaactggtgttccttgcgctgcagttcttgacgttgttacagaggctggaagggattggttgcttcttggtgaagtgccaggacaggacttgctttctagtcatctggcaccagctgagaaggtgtctataatggctgatgccatgcgaagacttcacacacttgatcctgccacatgtcctttcgatcaccaggctaagcataggattgagagggctaggactagaatggaagcaggtttggtggaccaagacgatcttgatgaggagcatcaaggacttgctccagctgaacttttcgcaaggctcaaagctaggatgccagatggagaggatcttgttgtgactcacggagatgcttgcttgcccaacatcatggttgagaatgggaggttctctggattcatcgactgtggtagactcggagttgcagataggtaccaggacattgcacttgcaactcgtgacatcgctgaggaacttggaggtgaatgggcagatcgtttccttgtcctctatgggattgctgcacctgattcacagcgtatcgcattctaccgactgctcgatgagttcttctgaaccggtgaaatcaccagtctctctctacaaatctatctctctctattttctccataataatgtgtgagtagttcccagataagggaattagggttcttatagggtttcgctcacgtgttgagcatataagaaacccttagtatgtatttgtatttgtaaaatacttctatcaataaaatttctaattcctaaaaccaaaatccagtactaaaatccagatcacctaaagtccctatagatctttgtcgtgaatataaaccagacatgagacgactaaacctggagcccagacgccgttcgaagctagaagtaccgcttaggcaggaggccgttagggaaaagatgctaaggcagggttggttacgttgactcccccgtaggtttggtttaaatatgataaagtggacgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 18 - DNA, 12909 bp, synthetic construct
  source 1..12909: mol_type = other DNA
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagttttacggccggccgaagctttttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgatttccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtctagatatttttacaacaattaccaacaacaacaaacaacaaacaacattacaattactatttacaattacacccgggtctagacatatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcagcatggacgagctgtacaagtaagagctcaccggtcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattgcggccggccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgactagtggtacaattctcaacacaacatatacaaaacaaacgaatctcaagcaatcaagcattctacttctattgcagcaatttaaatcatttcttttaaagcaaaagcaattttctgaaaattttcaccatttacgaacgatagcctaggatgattgaacaggatggtcttcatgctggatctccagctgcatgggttgaacgactctttgggtatgactgggcacaacagaccataggttgctctgatgctgcagttttcaggcttagtgcacaagggagacctgttctcttcgtcaagactgacttgtccggagctttgaatgagcttcaggatgaggctgccagactttcatggcttgctacaactggtgttccttgcgctgcagttcttgacgttgttacagaggctggaagggattggttgcttcttggtgaagtgccaggacaggacttgctttctagtcatctggcaccagctgagaaggtgtctataatggctgatgccatgcgaagacttcacacacttgatcctgccacatgtcctttcgatcaccaggctaagcataggattgagagggctaggactagaatggaagcaggtttggtggaccaagacgatcttgatgaggagcatcaaggacttgctccagctgaacttttcgcaaggctcaaagctaggatgccagatggagaggatcttgttgtgactcacggagatgcttgcttgcccaacatcatggttgagaatgggaggttctctggattcatcgactgtggtagactcggagttgcagataggtaccaggacattgcacttgcaactcgtgacatcgctgaggaacttggaggtgaatgggcagatcgtttccttgtcctctatgggattgctgcacctgattcacagcgtatcgcattctaccgactgctcgatgagttcttctgaaccggtgaaatcaccagtctctctctacaaatctatctctctctattttctccataataatgtgtgagtagttcccagataagggaattagggttcttatagggtttcgctcacgtgttgagcatataagaaacccttagtatgtatttgtatttgtaaaatacttctatcaataaaatttctaattcctaaaaccaaaatccagtactaaaatccagatcacctaaagtccctatagatctttgtcgtgaatataaaccagacatgagacgactaaacctggagcccagacgccgttcgaagctagaagtaccgcttaggcaggaggccgttagggaaaagatgctaaggcagggttggttacgttgactcccccgtaggtttggtttaaatatgataaagtggacgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

(19) United States
(12) Patent Application Publication
(10) Pub. No.: US 2024/0352473 A1
(43) Pub. Date: Oct. 24, 2024

(54) SEQUENCES AND PROMOTERS FOR USE IN PLANT CELLS AND METHODS OF MAKING AND USING SUCH SEQUENCES

(71) Applicant: Futuragene Israel Ltd., Rehovot (IL)

(72) Inventors: Dror Avisar, Ramat Gan (IL); Shelly Azulay, Ness Ziona (IL)

(21) Appl. No.: 18/685,643

(22) PCT Filed: Aug. 23, 2022
(86) PCT No.: PCT/IB2022/057897

§ 371 Date: Feb. 22, 2024

Related U.S. Application Data
(60) Provisional application No. 63/236,474, filed on Aug. 24, 2021.

(51) Int. Cl. C12N 15/82; C12N 9/22; C12N 15/11

(52) U.S. Cl. CPC C12N 15/8216; C12N 9/22; C12N 15/11; C12N 15/8203; C12N 2310/20; C12N 2710/00043

(57) ABSTRACT

The present disclosure is directed to a novel sequence constructed from viral elements for use as a transgenic promoter; for example, in transgenic plants. More specifically, the present disclosure is directed to a chimeric transgenic promoter sequence comprising a portion derived from the Figwort Mosaic Vims (FMV/FiMV) genome and a portion derived from the Cassava Vein Mosaic Virus (CsVMV) genome. The present disclosure provides methods and compositions for the making and using such a transgenic promoter.

### FIELD OF THE INVENTION

The present invention relates in general to nucleic acid sequences which may serve as promoters for transgenic expression. More specifically, the invention relates to sequence elements derived from viral promoters and the use of combinations of these sequence elements to express coding sequences or functional RNAs in plants.

### SEQUENCE LISTING

This application contains a Sequence Listing that has been submitted electronically as an XML file named “30407-0016W01_SL_ST26.xml.” The XML file, created on Aug. 22, 2022, is 55,300 bytes in size. The material in the XML file is hereby incorporated by reference in its entirety.

### BACKGROUND

One of the goals of plant genetic engineering is to produce plants with ergonomically preferable characteristics or traits, and for this aim-enhancing or reducing the expression level of a gene product (or products) or of functional RNAs. Such changes in expression commonly require the use of a non-endogenous promoter.

Whereas for some plants and crops there is a wide set of promoters available for transgenic use, others, such as Eucalyptus, have but a few non-endogenous promoters which are well-characterized to be functional, even for use for constitutive transgenic expression. Thus, constructing promoters for such crops is valuable.

### SUMMARY

In one aspect, the present disclosure provides (I) a nucleic acid sequence which comprises (i) a transcriptional regulatory element derived from the sub-genomic transcript (Sgt) promoter of the Figwort Mosaic Virus (FMV, FiMV), which does not include the promoter's TATA portion, and a (ii) transcription regulatory element derived from the genome of Casava Vein Mosaic Virus (CsVMV) promoter which does include a TATA portion, or (II) a nucleic acid sequence that comprises sequences substantially similar to the (i) and (ii) sequences described above.

In an additional aspect, the present disclosure provides bacteria-clone propagated plasmids which include the nucleic acid sequence described above, and which can function as expression vectors in plant cells.

The present disclosure also provides a transformed plant cell having in its genome the nucleic acid sequence described above, as well as transgenic plants or seeds including such plant cells.

### BRIEF DESCRIPTION OF DRAWINGS

FIG. 1

Schematically depicts a non-limiting nucleic acids construct comprising a pFSgt-CsVMV sequence. The illustrated construct (4478 bp long) comprises the pFSgt-CsVMV of SEQ ID NO: 1 cloned into the pUC57 vector backbone.

FIG. 2

Schematically depicts a non-limiting nucleic acids construct comprising a pFSgt-BRRV sequence: The DNA construct (4335 bp long), comprises the pFSgt-BRRV SEQ ID NO: 2, cloned into the pUC57 vector backbone.

FIG. 3

Schematically depicts the construct comprising a pFSgt-PFIt sequence: The DNA construct (4344 bp long), comprises SEQ ID NO: 3 cloned into the pUC57 vector backbone.

FIG. 4

Schematically depicts the construct comprising a CaMV 35S sequence: The DNA construct (4367 bp long), comprises SEQ ID NO: 4, cloned into the pUC57 vector backbone.

FIG. 5

Shows fluorescence microscopy imaging of GFP from protoplast cells comprising the four constructs described in FIG. 1-4: Specifically, microscopic images show GFP fluorescence from Eucalyptus protoplast cells transformed with the expression constructs described in Example 3, and observed 24 hours post transformation, as an indicator of expression. Chlorophyll auto-fluorescence is seen in red.

FIG. 6

Fluorescence quantification from the expression experiment shown in FIG. 5: Quantification of GFP fluorescence intensity from the transformed Eucalyptus protoplasts shown in FIG. 5. Intensity quantified and normalized for cell size, using ImageJ. Mean±SEM.

FIG. 7A

Schematically depicts the binary vector construct comprising pFSgt-CsVMV (SEQ ID NO: 1) operably linked to mCherry reporter: The DNA construct (12830 bp long), is provided in full as SEQ ID NO: 17 (pFSgt-CsVMV synthetic vector).

FIG. 7B

Schematically depicts the binary vector construct comprising pFSgt-CsVMV (SEQ ID NO: 1) followed by a downstream Omega (Om) sequence operably linked to mCherry reporter: The DNA construct (12909 bp long), is provided in full as SEQ ID NO: 18 (pFSgt-CsVMV synthetic vector+TMV omega 5′ UTR).

FIG. 8A-8H

Fluorescence images showing mCherry fluorescence (expression) in Eucalyptus explants/callus transformed with constructs comprising pFSgt-CsVMV promoter sequences. FIG. 8A, 8C, 8E and 8G show light images of cells transformed with either the binary vector described in FIG. 7A (FIG. 8A, 8C) OR with a binary vector described in FIG. 7B (FIG. 8E, 8G). FIG. 8B, 8D, 8F and 8H show the corresponding mCherry emissions—shown in red, corresponding to FIG. 8A, 8C, 8E and 8G, respectively.

FIG. 9

Shows the sequences disclosed herein.

### DETAILED DESCRIPTION

Described herein are sequences, compositions and methods useful for driving expression of a transgene. Specifically described herein are sequences functional as promoters and expression vectors which carry them, for use in plant cells. As detailed in the experimental examples below, a pFSgt-CsVMV sequence was used for transgenic expression and found to be functional in plant cells.

We designed a non-naturally occurring nucleic acid sequence we call pFSgt-CsVMV based on sequences derived from genomes of two viruses which infect plants: the Figwort Mosaic Virus (FMV/FiMV, NCBI ID: 10649) and the Cassava Vein Mosaic Virus (CsVMV, NCBI ID: 38062).

Nucleic acid sequences described herein generally include (1) an isolated non-naturally occurring nucleic acid sequence comprising (i) a first sequence substantially similar to a portion of the Figworts Mosaic Virus (FMV, FiMV) sub-genomic transcript (Sgt/sg) promoter (sgFiMV, SEQ ID NO: 16), which lacks a TATA box, and (ii) a second, TATA-including portion, substantially similar to a fragment of the Casava Vain Mosaic Virus (CsVMV) genome. In addition to the pFSgt-CsVMV nucleic acid sequence, (2) a cell harboring the aforementioned isolated nucleic acid sequence, (3) a plant comprising at least one cell harboring the aforementioned nucleic acid sequence, and (4) a method of making a transgenic plant using the aforementioned nucleic acid sequence also are provided.

The pFSgt-CsVMV sequence may be constructed in several ways, but more specifically encompasses (from 5′ to 3′ direction):
(1) a first portion of the sgFiMV promoter region (the sgFiMV promoter being SEQ ID NO: 16—nucleotides (nt.) 5063-5363 of the FMV reference genome; NC 003554.1)—but such a portion which lacks the sgFiMV TATA Box (located in nt. 5287-5293) and its surrounding nucleotides (10 nucleotides from each side), or a similar sequence thereof. This first portion is followed by
(2) A sequence portion derived from the Cassava Vain Mosaic virus (CsVMV) genome (and specifically more than 200 nt.) from the segment 7162-7604 Of NCBI ID U59751.1, which is provided as SEQ ID NO: 6, or a similar sequence thereof).

A non-limiting example of a nucleic acid sequence as described herein is the pFSgt-CsVMV of SEQ ID NO: 1 which is comprised of a (i) sequence (of over 200 nucleotide bases) derived from the sgFiMV transcript promoter lacking the FMV-derived TATA box, and surrounding nucleotides; and (ii) a sequence (over 400 nucleotide bases long) from a CsVMV promoter—including the CsVMV derived TATA box.

The CsVMV sequence portion is proximal to the Transcription start site (TSS) and to an operably linked transcribed sequence-when the pFSgt-CsVMV is used as a transgenic promoter.

In more detail: the pFSgt-CsVMV of SEQ ID NO: 1 comprises (a) a nucleotide sequence derived from the FMV genome, specifically the SEQ ID NO: 5—which lacks the ‘proximal promoter’ (i.e. proximal to the TSS) portion of that native FMV promoter; and (b) a nucleotide sequence from the CsVMV genome (specifically SEQ ID NO: 6) representing a proximal promoter portion of the CsVMV promoter, and may include a UTR portion (SEQ ID NO: 8).

The SEQ ID NO: 6 used in the construction of SEQ ID NO: 1—differs in 1 base pair from the (GenBank Seq. ID U59751.1) CsVMV genome, as the guanine (G) at position 7234 is replaced with adenine (A), to eliminate the SacI restriction site (GAGCTC to AAGCTC) for simplicity in subsequent cloning The comparable non-mutated Cassava sequence, as in the GenBank Seq. ID U59751.1 is provided as SEQ ID NO: 7.

Another exemplary construction of a pFSgt-CsVMV sequence is provided as SEQ ID NO: 9.

pFSgt-CsVMV is said to comprise a first and second portions. When used to drive expression of a transgene, the first portion (derived from the sub-genomic transcript promoter of FMV, SEQ ID NO: 16) may be described as distal and the second (derived from CsVMV) as proximal in respect to a transcribed sequence—or more accurately in respect to the transcription start site (TSS). Notably, these two portions are sometimes referred to as the “core promoter” which includes the TATA and “regulatory module” (the upstream portion), such as the pFSgt-CsVMV may be described as (1) a nucleic acid sequence comprising a first sequence substantially similar to a regulatory module of the sub genomic transcript promoter of FMV and a second sequence substantially similar to a core promoter of CsVMV, or alternatively as (2) a hybrid promoter sequence comprising (i) the regulatory module of the sub-genomic transcript promoter of FMV promoter or a substantially similar sequence thereof, and (ii) the core promoter of CsVMV or a substantially similar sequence thereof.

The nucleic acid sequence described herein may be used as a promoter for diverse aims—including, but not limited to—expression of protein-coding sequences or for the transcription of functional RNAs. A functional RNA is thought of in the art as a transcribed RNA molecule with cell-biology functions, such as, for example: an mRNA, a miRNA, a tRNA, a dsRNA, a triplex RNA, a ribozyme, a long non-coding RNA, a snoRNA, snRNA, an enhancer ncRNA, a Piwi-interacting RNA (piRNA), CRISPR guideRNA (gRNA), CRISPR RNA (crRNA), trans-activating CRISPR RNA (tracrRNA), donor-RNA and sponge RNA.

Non-limiting examples for protein-coding sequences (e.g., genes) to be expressed include genes useful in providing desirable traits in cultivated plants such as enhanced biomass, resistance to pests (such as insects, fungus and other pathogenic agents) enhanced tolerances to herbicides, environmental tolerances to a biotic stress such as drought, and enhanced processability of plant-derived product(s) including wood lignin.

The present disclosure describes a method of expressing a transcribed element in a plant by steps inducing introducing the pFSgt-CsVMV into a plant cell.

Optionally and preferably such a method includes further selection for plant cells positive for the transgenic sequence or selection of transgenic plants, and based on desired phenotype(s). In such a method—introducing the pFSgt-CsVMV into a plant genome may be achieved by genome editing methods selected from TALEN or CRISPR, and specifically by CRISPR Site Directed Integration (SDI).

For the sake of clarity: two unrelated viruses referred to as “FMV” are known in the ART: the focus of this disclosure are sequences from the double stranded DNA (dsDNA) Figwort Mosaic Virus (FMV; NCBI ID: 10649): a non-enveloped virus. The genome of FMV; NCBI ID: 10649 was provided by Shepherd (NC_003554.1 GI: 20143424), as described above. Another, unrelated virus is the Fig mosaic Virus (emaravirus) (FMV; NCBI ID: 54539) a segmented, negative sense, single-stranded RNA virus that is the causal agent of Fig Mosaic Disease (FMD) in fig plants. The latter is not of relevance to the present disclosure.

In the field of plant transgenics a promoter is commonly selected for its ability to direct transcription (in a transformed cell or plant) of a desired expressed sequence (E.g., coding sequence or functional RNA) in an expression-level sufficient to provide a desired biological function or effect. The nucleic acids described herein are of specific value since redundant use of the same promoter several times in the same cell dilutes its potency.

pFSgt-CsVMV may be used in parallel to another high expressing promoter, and the co-expression would not be subjected to the same dilution affect as would be rendered when a single promoter is used for both transgenes in the same cell.

The methods and compositions described herein may be applied to any monocot and dicot plant or plant cell, without limitation, and in order to provide gene expression.

Relevant example plants include woody plants (perennial plant having an elongated hard lignified stem; i.e., trees), such as Eucalyptus, poplar, pine, fir, spruce, acacia, sweet gum, ash, birch, oak, teak, mahogany, sugar and Monterey, nut trees, E.g., walnut and almond, and fruit trees, E.g., apple, plum, cherry, citrus and apricot. Other notable examples are trees and plants can be alfalfa, artichoke, arugula, asparagus, avocado, banana, barley, beans, beet, blackberry, blueberry, broccoli, Brussel-Sprouts, cabbage, canola, cantaloupe, carrot, cassava, cauliflower, celery, cilantro, coffee, corn, cotton, cucumber, duckweed, eggplant, endive, escarole, fennel, gourd, Indian mustard, safflower, olive, rice, barley, sugarcane, wheat, duckweed. Eucalyptus and related plants are of specific interest. Relevant tree subtypes amendable for use with the nucleic acids described herein include but are not limited to Eucalyptus and pine species, for example, Eucalyptus (such as Eucalyptus grandis) or its hybrids, or Pinus subtypes. For example: Eucalyptus alba, Eucalyptus bancroftii, Eucalyptus botryoides, Eucalyptus bridgesiana, Eucalyptus calophylla, Eucalyptus camaldulensis, Eucalyptus citriodora, Eucalyptus cladocalyx, Eucalyptus coccifera, Eucalyptus curtisii, Eucalyptus dalrympleana, Eucalyptus deglupta, Eucalyptus delagatensis, Eucalyptus diversicolor, Eucalyptus dunnii, Eucalyptus ficifolia, Eucalyptus globulus, Eucalyptus gomphocephala, Eucalyptus gunnii, Eucalyptus henryi, Eucalyptus laevopinea, Eucalyptus macarthurii, Eucalyptus macrorhyncha, Eucalyptus maculata, Eucalyptus marginata, Eucalyptus megacarpa, Eucalyptus melliodora, Eucalyptus nicholii, Eucalyptus nitens, Eucalyptus nova-angelica, Eucalyptus obliqua, Eucalyptus occidentalis Eucalyptus obtusiflora, Eucalyptus oreades, Eucalyptus pauciflora, Eucalyptus polybractea, Eucalyptus regnans, Eucalyptus resinifera, Eucalyptus robusta, Eucalyptus rudis, Eucalyptus saligna, Eucalyptus sideroxylon, Eucalyptus stuartiana, Eucalyptus tereticornis, Eucalyptus torelliana, Eucalyptus urnigera, Eucalyptus urophylla, Eucalyptus viminalis, Eucalyptus viridis, Eucalyptus wandoo, and Eucalyptus youmanni, OR Pinus banksiana, Pinus brutia, Pinus caribaea, Pinus clasusa, Pinus contorta, Pinus coulteri, Pinus echinata, Pinus eldarica, Pinus ellioti, Pinus jeffreyi, Pinus lambertiana, Pinus massoniana, Pinus monticola, Pinus nigra, Pinus palustrus, pinus pinaster, Pinus ponderosa, Pinus radiata, Pinus resinosa, Pinus rigida, Pinus serotina, Pinus strobus, Pinus sylvestris, Pinus taeda, Pinus virginiana, OR Abies amabilis, Abies balsamea, Abies concolor, Abies grandis, Abies lasiocarpa, Abies magnifica, Abies procera, Chamaecyparis lawsoniona, Chamaecyparis nootkatensis, Chamaecyparis thyoides, Juniperus virginiana, Larix decidua, Larix laricina, Larix leptolepis, Larix occidentalis, Larix siberica, Libocedrus decurrens, Picea abies, Picea engelmanni, Picea glauca, Picea mariana, Picea pungens, Picea rubens, Picea sitchensis, Pseudotsuga menziesii, Sequoia gigantea, Sequoia sempervirens, Taxodium distichum, Tsuga canadensis, Tsuga heterophylla, Tsuga mertensiana, Thuja occidentalis, Thuja plicata.

As used herein the words “gene expression” can be taken to be understood as referring to the well-known methods for expressing a nucleic acid or amino acid of interest, weather that be a peptide, a protein or a functional RNA of sorts. Examples of a functional RNAs have been provided above.

As used herein, the term “nucleic acid” refers to a single or double-stranded polymer of deoxyribonucleotide or ribonucleotide bases read from the 5′ to the 3′ end. The “nucleic acid may also optionally contain non-naturally occurring or altered nucleotide bases that permit correct read Through by a polymerase and do not reduce expression of a polypeptide encoded by that nucleic acid.

As used herein, the term “DNA” or “DNA molecule” refers to a double-stranded DNA molecule of genomic or synthetic origin, i.e., a polymer of deoxyribonucleotide bases or a polynucleotide molecule, read from the 5′ (upstream) end to the 3′ (downstream) end.

As used herein, the term “DNA sequence” refers to the nucleotide sequence of a DNA molecule. The nomenclature used herein is that required by Title 37 of the United States Code of Federal Regulations § 1.822 and set forth in the tables in WIPO Standard ST.25 (1998), Appendix 2, Tables 1 and 3.

It is common expectation in the field of transgenic promoters to assume—for promoter and regulatory nucleic acid elements over a certain length—that not all nucleotides exert a measurable functional effect. Thus, a sequence which is substantially similar—e.g., a sequence with at least 80% identity, or of 90% identity, or at least 95% identity, or at least 98% identity, or at least 99% identity—will probably have the same functional attributes—I.e.: a promoter with a substantially similar sequence will have a similar expression pattern.

In molecular biology, the term “transcription start site” (TSS) is the location on a DNA strand where the first nucleotide is transcribed into RNA. Notably, it is difficult to determine the exact position of the TSS using bioinformatics, but experimental methods can be used to locate it, notably high throughput sequencing.

The term TATA box (or Goldberg-Hogness box) is a DNA consensus sequence found in a portion of eukaryotic core-promoter regions transcribed by RNA polymerase II. It a sequence containing the consensus sequence 5′-TATA (A/T)A(A/T)-3′, and located ˜25-35 base pairs upstream to a transcription start site, from which transcription may be detected.

The portion of a promoter which is closer (proximal) to the TSS is said to be the proximal promoter (E.g, a ˜200-250 base pairs stretch upstream of the TSS, containing primary regulatory elements). Concordantly, a distal portion of the promoter is a sequence upstream yet separated from the TSS and that may contain additional regulatory elements.

The (i) FMV sub genomic transcript promoter and the (ii) CsVMV promoter are both TATA-box including promoters. The portion of the FMV sub genomic transcript promoter described as ID NO. 5 lacks the TATA-box sequences, and surrounding (proximal promoter) sequences.

A coding sequence (CDS) is a term of art describing a DNA encoding a gene product such as protein.

DNA elements to be used to comprise transgenic promoters by tools of molecular biology may be obtained by polymerase chain (PCR) amplification from a wide set of sources such as genomic sequences, plasmids and libraries of nucleic acid sequences, many of which are publicly available. Alternatively, such DNA elements can be synthesized chemically based on sequences described electronically in depositories. Such DNAs can be synthesized either in full or in portions which are subsequently conjugated to generate the complete promoter.

The term “recombinant DNA” or “recombinant nucleotide sequence” is understood in the art to mean DNA that contains a genetically engineered modification through manipulation via mutagenesis, restriction enzymes, ligation, and the like.

The phrase “functional in a plant” such as for a nucleic acid that functions as a promoter can be taken to refer to the ability of that nucleic acid to drive expression in a plant nucleus when operably linked to a sequence to be expressed. A promoter and expressed sequence are operably linked—when joined as part of the same nucleic acid molecule and suitably positioned and oriented for transcription to be initiated.

Nucleic acid sequences described herein may function as a promoter in plant cells. For example, we examined expression in plants of a coding sequence operably linked following the pFSgt-CsVMV nucleic acid sequence with or without an intervening omega 5′ UTR sequence element (“Om”); a nucleic acid sequence transcribed into omega leader of TMV RNA, of SEQ ID NO: 11. The omega leader of tobacco mosaic virus (TMV) may enhance translation of foreign RNAs both in vivo and in vitro; it may act as a translational enhancer in various cell types and different cell-free translational systems, upon transcription. The enhancement effect may be due to a stable compact structure of the omega sequence that avoids degradation.

For some applications it is preferred to introduce a functional recombinant DNA at a non-specific location in a plant genome, which is achieved by random genomic integration. In special cases it is preferable to insert a recombinant DNA construct by site-specific integration. Both may be relevant to the methods described herein. Site-specific recombination systems include cre-10x as disclosed in U.S. Pat. No. 4,959,317 and FLP-FRT as disclosed in U.S. Pat. No. 5,527,695, and using site directed integration using CRISPR genome editing methods as disclosed in China patent application publication CN 107,142,282.

Several genome editing methods are known in the art. CRISPR genome editing is the method of using the CRISPR-Cas system (derived from the prokaryotic acquired immune system) to enable site directed changes as well as site-selected insertions of larger elements into genomes. The nucleic acids described herein may be combined with CRISPR use in several ways: (1) CRISPR may be used to introduce a promoter as described herein into a genome of a plant to drive the expression of an endogenous or of an exogenous sequence; (2) a promoter as described herein may be used to drive the expression of an element of a CRISPR genome editing system (I.e.: The Cas protein, guide RNA, template RNA and the like); or (3) a promoter as described herein may be used for the expression of an element of a different CRISPR-based system (e.g., CRISPR based genome-tagging or RNA-editing).

Adaptation of the CRISPR system to eukaryotes may be found in U.S. application Ser. No. 15/981,807 (University of California—Berkeley) and U.S. Pat. No. 8,697,359 (Broad Institute), and plant use is explicitly provided in several applications such as US Publication No. US 2020/0299717 (Ceres Inc.)

Numerous methods for introducing foreign genes into plants are known and can be used to insert a functional polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993).

The methods chosen vary with the host plant, and include chemical transfection methods such as calcium phosphate, microorganism-mediated gene transfer such as Agrobacterium (Horsch et al., Science 227:1229-31 (1985)), electroporation, micro-injection, and biolistic bombardment.

Expression cassettes and vectors and in vitro culture methods for plant cell or tissue transformation and regeneration of plants are known and available. See, e.g., Gruber et al., “Vectors for Plant Transformation,” in Methods in Plant Molecular Biology and Biotechnology, supra, pp. 89-119. The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722), and ballistic particle acceleration (U.S. Pat. No. 4,945,050). Agrobacterium mediated maize transformation (U.S. Pat. No. 5,981,840); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crosswa et al., (1986) Mol. Gen. Genet. 202:179-185).

The most widely utilized method for introducing an expression vector into plants is based on the natural transformation system of Agrobacterium. Agrobacterium (Agro) tumefaciens (A. tumefaciens) and A. rhizogenes are plant pathogenic soil bacteria, which genetically transform plant cells. Systems and methods for Agrobacterium-mediated gene transfer are provided in Gruber, et al, supra; Miki, et al., supra; and Moloney, et al., (1989) Plant Cell Reports 8:238. Similarly, the gene can be inserted into the T-DNA region of a Ti or Ri plasmid derived from A. tumefaciens or A. rhizogenes, respectively. The compatible NOS promoter and terminator are present in the plasmid pARC2 (ATCC Accession No. 67238). The virulence (vir) gene from either the Ti or Ri plasmid along with T-DNA portion, or via a binary system where the vir gene is present on a separate vector are known and referenced in U.S. Pat. No. 5,262,306. All the references above are incorporated by reference herein in their entirety. Monocot transformation is provided in EPO No. 604 662.

In combination with a promoter as described herein, a (i) selection marker or (ii) screenable marker may be used. For example,
(i) Commonly used selective marker genes include those conferring resistance to antibiotics such as kanamycin (nptl), hygromycin B (aph IV) and gentamycin (aac3 and aacC4) or resistance/tolerance to herbicides such as glufosinate (bar or pat), glyphosate (EPSPS), and AMPA (phno). EPSPS as referred to herein (a cp4 epsps (aroA: CP4)) is a herbicide tolerant form of 5-enolpyruvulshikimate-3-phosphate synthase (EPSPS) enzyme, which decreases binding affinity for glyphosate, thereby conferring increased tolerance to glyphosate herbicide. Examples of such selectable markers are illustrated in U.S. Pat. Nos. 5,550,318; 5,633,435; 5,780,708 and 6,118,047.
(ii) Screenable markers, which provide an ability to visually identify transformants and expression moreover to the green fluorescent protein (GFP) and mCherry used as indicators of expression and expression level in the examples below include: beta-glucuronidase or uidA gene (GUS) for which various chromogenic substrates are known. Fluorescence such as that acquired by microscopic imaging and measured is most commonly presented by (relative and) arbitrary units [A.u.].

In order to quantify images from florescent microscopy and assess the overall quantity of an expressed protein—tools and algorithms may be used which assume florescence is linearly proportional to protein quantity. The ImageJ software (Rasband, W. S., ImageJ, published by U. S. National Institutes of Health, Bethesda, Maryland, USA; has been used in this work—and is in the public domain—not subjected to copyright protection).

The term “isolated DNA molecule” can be taken to be a DNA molecule at least partially separated from other molecules normally associated with it in its native state. In one embodiment, the term “isolated” is also used herein in reference to a DNA molecule that is at least partially separated from nucleic acids which normally flank the DNA molecule in its native state. Thus, DNA molecules fused to regulatory or coding sequences with which they are not normally associated, for example as the result of recombinant techniques, are considered isolated herein. Such molecules are considered isolated even when present, for example in the chromosome of a host cell, or within a plasmid construct in solution. The term “isolated” in this context encompasses molecules not present in their native state or context.

As used herein “sequence identity” refers to the extent to which two optimally aligned polynucleotide or polypeptide sequences are identical throughout a window of alignment (e.g. window of alignment of nucleotides or amino acids). And wherein “optimally aligned” is in accordance with the criteria on which the algorithm is based.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than a given percent of the reference sequence over the window of comparison). Optimal alignment of sequences for aligning a comparison window are well known to those skilled in the art.

BLAST (basic local alignment search tool) is known in the ART as a prominent algorithm and program for comparing primary biological sequence information, such as the amino-acid sequences of proteins or the nucleotides of DNA and/or RNA sequences. BLAST is a registered trademark of the NCBI National Library of Medicine (National Center for Biotechnology Information, U.S. National Library of Medicine, 8600 Rockville Pike, Bethesda MD, 20894 USA). For use in sequence comparison when working with genes, BLAST can locate common genes in two related species, and can be used to map annotations from one organism to another. An output of BLAST in its common use includes an indication for sequence identity and/or similarity as well as a statistic for the alignment probability in respect to a null hypothesis scenario. BLAST finds similar sequences, by locating short matches between the two sequences. This process of finding similar sequences is called seeding. It is after this first match that BLAST begins to make local alignments. While attempting to find similarity in sequences, sets of common letters, known as words, and the heuristic algorithm of BLAST locates all words between the sequence of interest and the hit sequence or sequences from the database. This result will then be used to build an alignment. These words must satisfy a requirement of having a score of at least the threshold T, when compared by using a scoring matrix.

For comparisons of protein sequences—the BLASTP is commonly used, and for the comparison of a DNA to a protein to which it may code BLASTX (translated nucleotide sequences; BLASTX version 2.0) and BLASTN version 2.0 for reciprocally, identifying polynucleotide sequences which may translate to a protein query.

Unless otherwise indicated, all numbers defined above, used in this specification are to be understood as being modified in all instances by the term “about”. Accordingly, unless indicated to the contrary, the numerical parameters set forth in this specification are approximations that may vary by up to plus or minus 5%.

High expression of a transgene in one or more target cell types or tissues is by default defined as compared to a control, E.g., such as a commonly used-in-the-art promoter. Additional controls that can be used for comparison to determine high or increased transgene expression by a regulatory element disclosed herein include vector alone, a minimal promoter from a viral source or sequences that do not commonly drive expression. High expression of a transgene may be within a cell, or in vivo, in vitro, and/or ex vivo.

There are a number of variations or promoter sequences and transformation methods that can be used to generate any of the plants described herein. For example:
Odell, J. T., Nagy, F., & Chua, N. H. (1985). Identification of DNA sequences required for activity of the cauliflower mosaic virus 35S promoter. Nature, 313 (6005), 810-812.
Acharya, S., Ranjan, R., Pattanaik, S., Maiti, I. B., & Dey, N. (2014). Efficient chimeric plant promoters derived from plant infecting viral promoter sequences. Planta, 239 (2), 381-396
Timko M P, Kausch A P, Castresana C, Fassler J, Herrera-Estrella L, Van den Broeck G, Van Montagu M, Schell J, Cashmore AR. Light regulation of plant gene expression by an upstream enhancer-like element. Nature. 1985 Dec. 12-18; 318 (6046):579-82. doi: 10.10381318579a0. PMID: 3865055.
Deepak Kumar et al. published in PLOS in 2011 “Development of Useful Recombinant Promoter and Its Expression Analysis in Different Plant Cells Using Confocal Laser Scanning Microscopy”
Acharya et al. Planta, 2014 “Efficient chimeric plant promoters derived from plant infecting viral promoter sequences”
Yoo S. D. Cho Y. J., Sheen J. “Arabidopsis mesophyll protoplasts: a versatile cell system for transient gene expression analysis” Nat Protocols 2007; 2 (7):1565-72. doi: 10.1038/nprot.2007.199

The compositions and methods described herein will now be illustrated by the following non-limiting Examples.

### EXAMPLES

### Example 1: Construction of Sequences to be Used as Promoters

Non-naturally occurring sequences to be used as promoters for constitutive expression in dicot plants were constructed based on DNA sequence-fragments from viral genomes:
1) SEQ ID NO: 1 (pFSgt-CsVMV), is a non-limiting example of a psgFiMV-CsVMV promoter containing 726 nucleotides—based on portions of two viral genomic sequences: a first portion derived from the genome of Figworts mosaic virus and a second portion derived from the genome of Casava Vein Mosaic Virus. The second portion (TATA-containing) is comparable to SEQ ID NO: 6 and SEQ ID NO: 8, as in Table No. 1 below.

TABLE NO. 1
sequence elements in SEQ ID NO: 1

| | Nucleotides of | | |
|---|---|---|---|
| | SEQ ID NO: 1: | SEQ ID NO | Derived from |
| a | 1-211 | SEQ ID NO: 5 | 5063-5273 nt promoter portion from the Figwort |
| | | | mosaic virus genome (GenBank Accession No. |
| | | | ′X06166.1) |
| b | 212-654 | SEQ ID NO: 6 | 7162-7604 nt promoter portion from the CsVMV |
| | | | genome (GenBank Accession No. U59751.1) |
| c | 655-726 | SEQ ID NO: 8 | 7605-7676 nt 5-UTR portion from the CsVMV |
| | | | genome (GenBank Accession No. U59751.1) |

2) SEQ ID NO: 2, is an example of a pFSgt-BRRV sequence (583 nucleotides). SEQ ID NO: 2 was synthesized and composed of the following DNA fragments:

TABLE NO. 2
sequence elements in SEQ ID NO: 2

| | Nucleotides of | | |
|---|---|---|---|
| | SEQ ID NO: 2: | SEQ ID NO | Derived from |
| a | 1-211 | SEQ ID NO: 5 | 5063-5273 nt promoter portion from the Figwort |
| | | | mosaic virus genome (GenBank Accession No. |
| | | | X06166.1) |
| b | 212-516 | SEQ ID NO: 10 | 5721-6025 nt enhancer portion from the Blueberry red |
| | | | ringspot virus (BRRV) genome (GenBank Accession |
| | | | No. AF404509.2) |
| c | 517-583 | SEQ ID NO: 11 | 2-68 nt Omega (Om) from the Tobacco mosaic virus |
| | | | (TMV) genome (GenBank Accession No. |
| | | | NC_001367.1) |

In respect to a canonical CsVMV genome (ID U59751.1; SEQ ID NO: 7)-the guanine (G) at position 7234 (which is within SEQ ID NO: 6) was replaced with adenine (A), to eliminate the Sad restriction site (GAGCTC to AAGCTC) for simplicity in cloning.

The synthesis of the pFSgt-CsVMV and pFSgt-BRRV sequences—later cloned for use in expression vectors—was done using the service of GeneWiz and verified by Sanger sequencing.

Two additional sequences were synthesized and later used as controls: (I) pFSgt-PFIt and CaMV 35S. The pFSgt-PFIt sequence includes portions from a peanut chlorotic streak (Caulimovirus) virus (PC1SV; NCBI: txid 35593) promoter with a (proximal, TATA-box-including portion) fragment from the Figworts mosaic virus promoter; and (II) the CaMV 35S promoter, commonly used in the art.

### Example 2: Cloning of Expression Constructs

In order to functionally assess the transcriptional activity of novel chimeric promoters in-vivo, four promoters (described above and listed below) were cloned into the pUC57 vector backbone upstream of a green fluorescent protein (GFP; GenBank Seq. ID X96418.1) coding sequence (CDS), followed by an A. tumefaciens NOS terminator (21790-21538 nt of GenBank Accession No. MK439386.1). The four promoters used were as follows:
(I) pFSgt-CsVMV promoter (SEQ ID NO: 1);
(II) pFSgt-BRRV promoter (SEQ ID NO: 2);
(III) pFSgt-PFIt promoter (SEQ ID NO: 3);
(IV) CaMV 35S promoter (SEQ ID NO: 4).

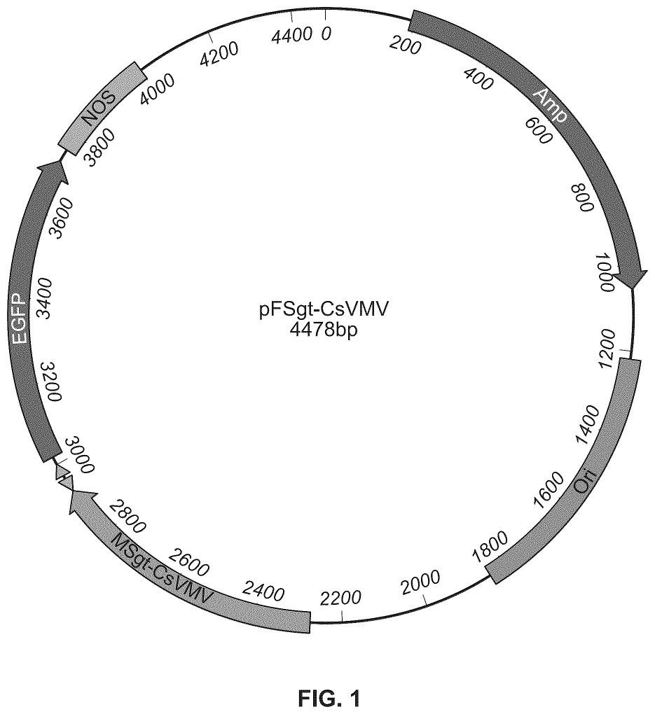

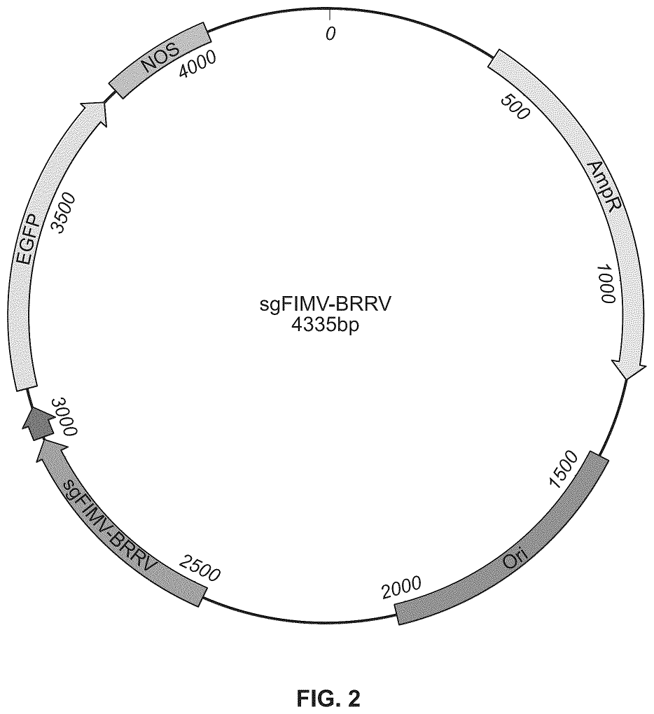

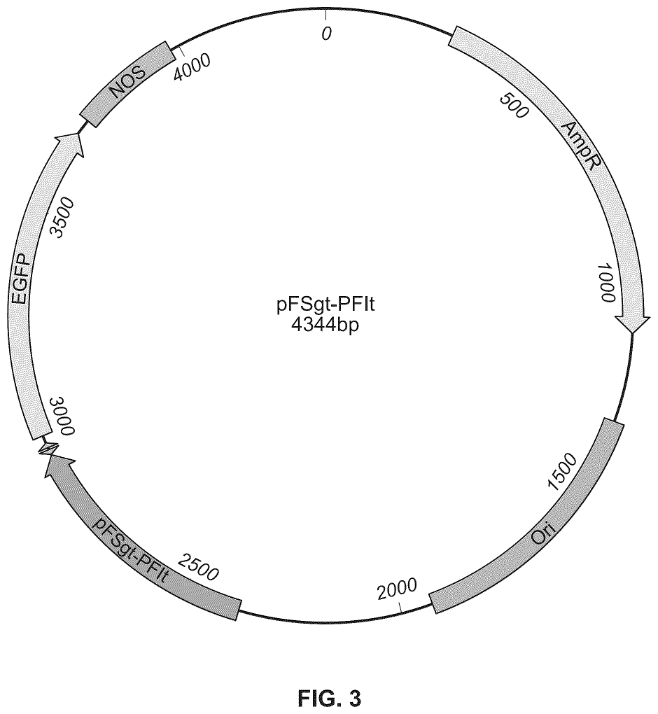

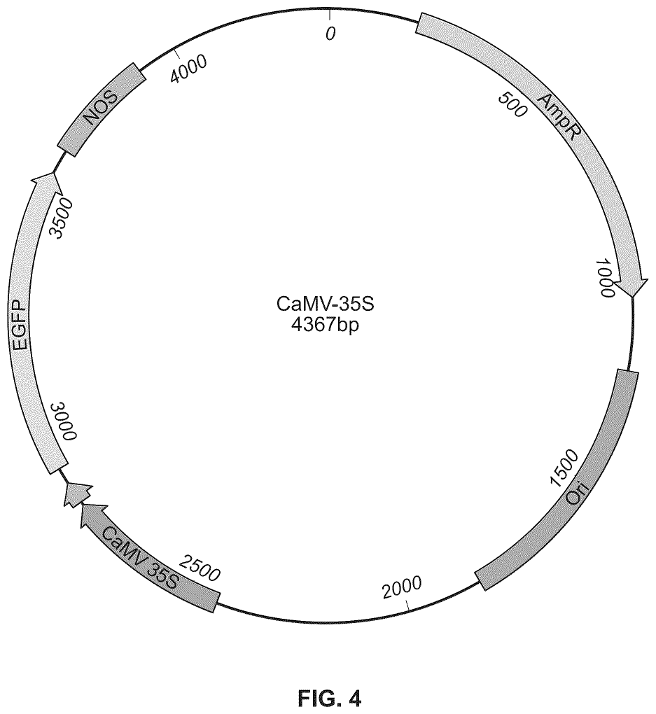

The pFSgt-CsVMV promoter was re-sequenced within the pUC57 vector backbone. A reverse-complement segment (of 800 nt) from Sanger sequencing is provided as SEQ ID NO: 9. All 4 constructs (the scheme of each is provided in FIGS. 1-4, respectively) were transformed into protoplasts:

Specifically, isolated protoplasts derived from plant leaves of Eucalyptus grandis were extracted and transformed (PEG transformation) as in Yoo et al., 2007, Nature Protocols, 2:1565-72. Protoplasts were incubated overnight at 25° C. in an osmotic solution, and GFP expression was observed 24 hours post transformation.

### Example 3: Protoplasts Expression Assay

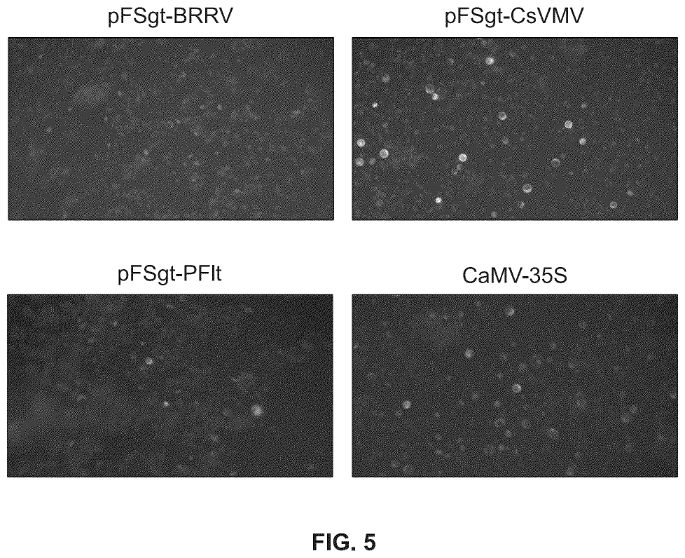

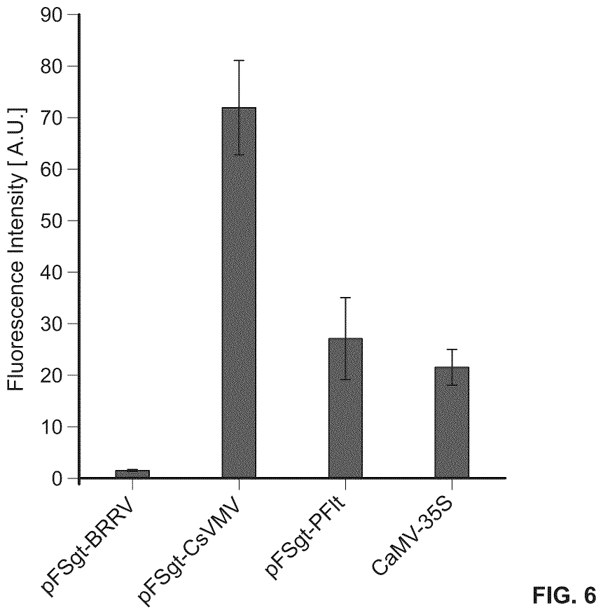

In order to assess mean expression level, transformed protoplasts were viewed under a fluorescent stereo microscope, and GFP intensity, as a proxy of expression level, was quantified (using ImageJ). FIG. 5 shows that, 24 hours post transformation, the pFSgt-CsVMV-GFP protoplasts had higher expression as compared to the protoplasts expressing the GFP reporter from a CaMV 35S promoter. By similar comparison, the pFSgt-BRRV-GFP, as compared to the CaMV 35S-GFP, was found to have relatively low expression levels. FIG. 6 provides a quantification of the GFP intensity visually observed in FIG. 5, as a proxy of expression level, indicating pFSgt-CsVMV-GFP protoplasts had over 3-fold higher expression as compared to the CaMV 35S-GFP control.

### Example 4: Cloning and Transformation Into Plants

In order to assess the expression profile of the new promoter in planta, Eucalyptus cells were transformed with a construct harboring the pFSgt-CsVMV promoter as described below.

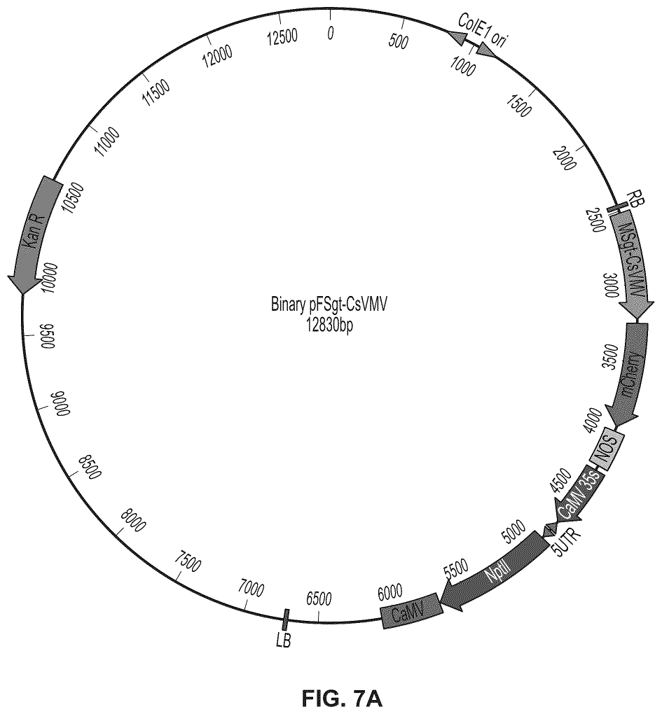

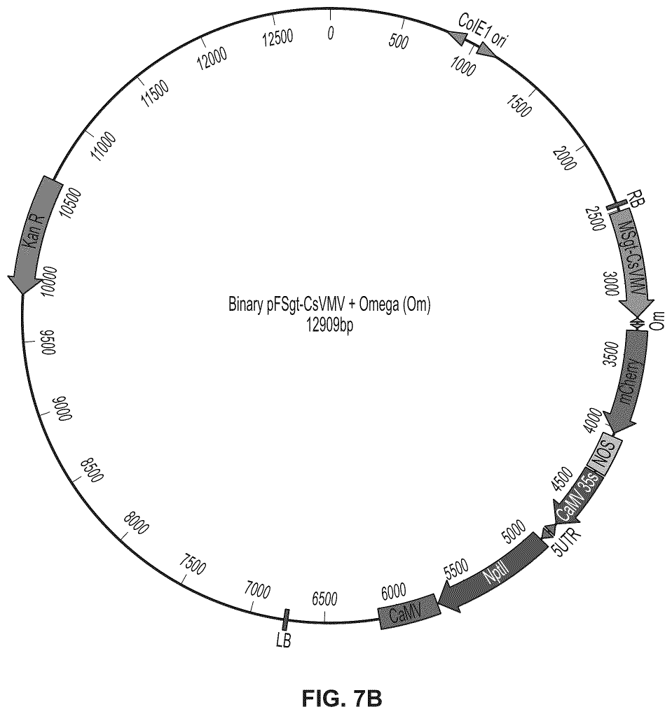

Specifically, DNA constructs encompassing the synthetic sequence of the pFSgt-CsVMV promoter, SEQ ID NO: 1, were generated by cloning the pFSgt: CsVMV into a pBI121 backbone, using HindIII-XbaI sites as in FIG. 7A. The pFSgt-CsVMV promoter of SEQ ID NO: 1 is followed by the CDS of mCherry and a NOS terminator (pFSgt-CsVMV-mCherry), SEQ ID NO:17. Construct as in FIG. 7B is essentially identical to the construct as in FIG. 7A, except that there is an Omega sequence downstream to SEQ ID NO: 1 and upstream to the mCherry sequence. Constructs were verified by Sanger sequencing, and transformed into plants essentially as described in Prakash et al., 2009, Phcog. Rev., 3:353-8: shoots of Eucalyptus were propagated in vitro on Murashige and Skoog medium (MS also called MSO or MS0 (MS-zero)) basal salt medium consisting of 3% (w/v) sucrose and 0.8% (w/v) agar. All in vitro plant materials were incubated at 25±2° C. for 16-h photoperiod with cool white fluorescent lamps with an intensity of 30 llEm−2 s−1. A. tumefaciens strain LB A 4404 harboring a binary vector pBI121 containing nptll gene was used for transformation. Agro bacterial culture collected at late log phase was pelleted and re-suspended in MS basal salt medium. Leaves from in vitro material were collected and used as explants for transformation experiments. Explants were pre-cultured on the MS regeneration medium supplemented with 0.5 mg/1 6-benzylaminopurine (BAP) and 0.1 mg/1 NAA for 2 d. Later, pre-cultured leaf explants were gently shaken in the bacterial suspension for 10 min and blotted dry on a sterile filter paper. Explants were then cultivated in medium under the pre-culture conditions for two days.

Following co-cultivation, explants (e.g., transformed with constructs encompassing the pFSgt-CsVMV-mCherry) were washed in MS liquid medium, blotted dry on a sterile filter paper, and transferred to MS regeneration medium containing 0.5 mg/l 6-benzylaminopurine and 0.1 mg/l 1-Naphthaleneacetic acid supplemented with 40 mg/l. kanamycin and 300 mg/l cefotaxime. After 4-5 weeks of culture, regeneration was observed and explants transferred to liquid elongation medium (MS medium supplemented with 0.5 mg/1 BAP, 40 mg/l kanamycin, and 300 mg/1 cefotaxime) on paper bridges. The elongated shoots (1.5-2 cm) were propagated on MS medium with 0.1 mg/1 BAP. Leaf segments were regenerated, and positive explants were grown on MS medium containing 0.04 mg/L BAP.

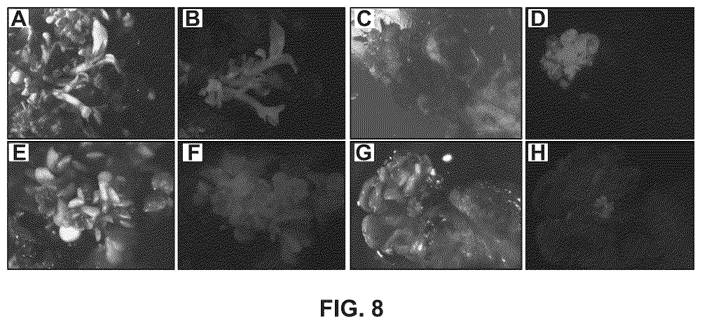

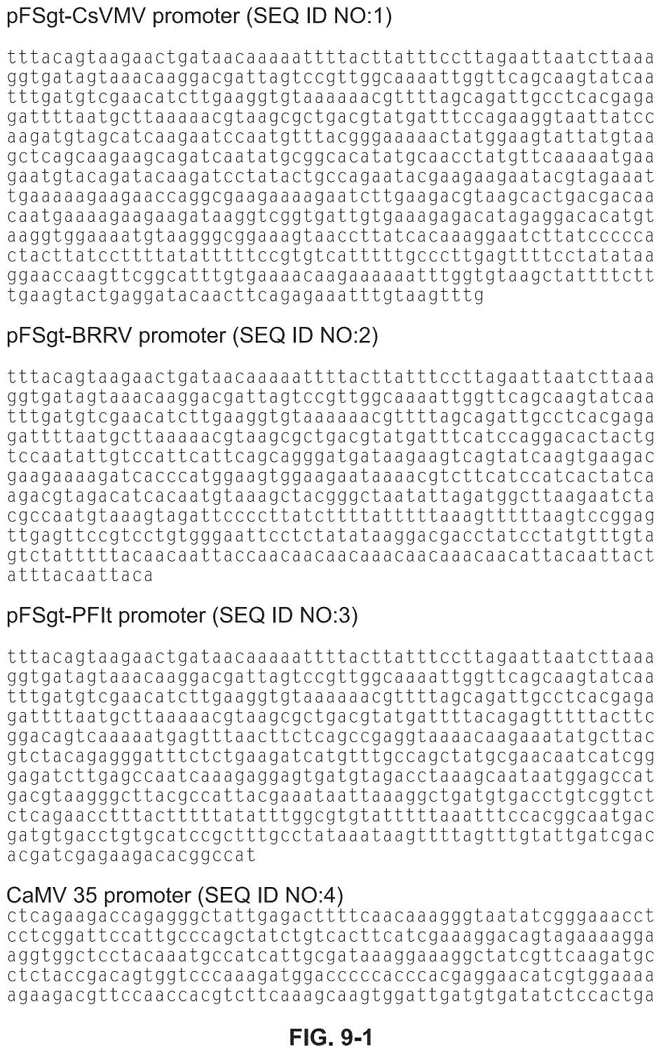

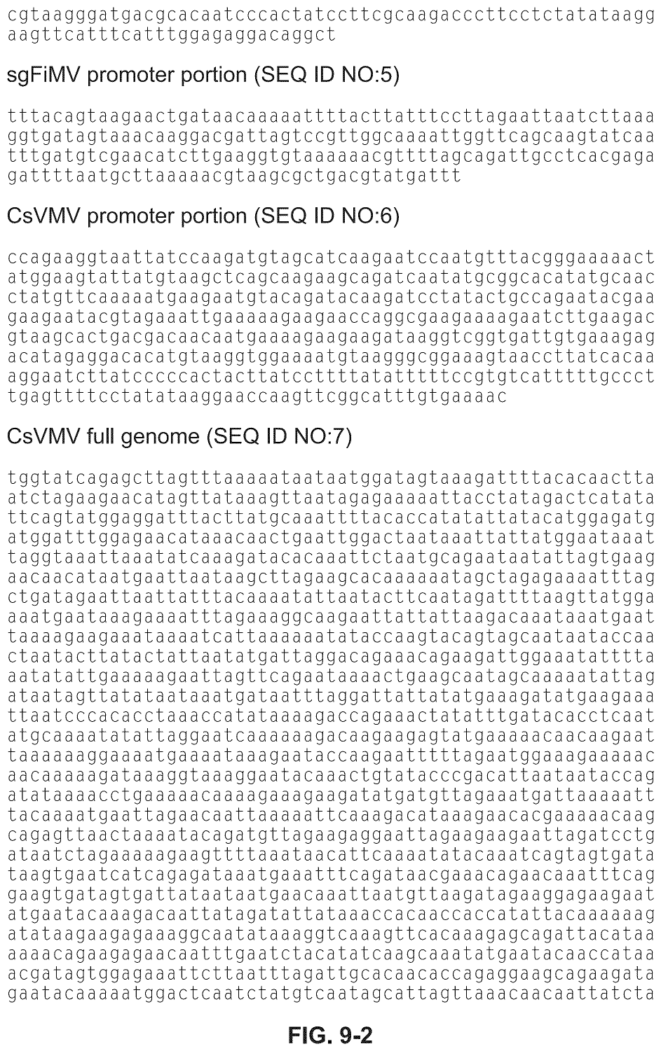

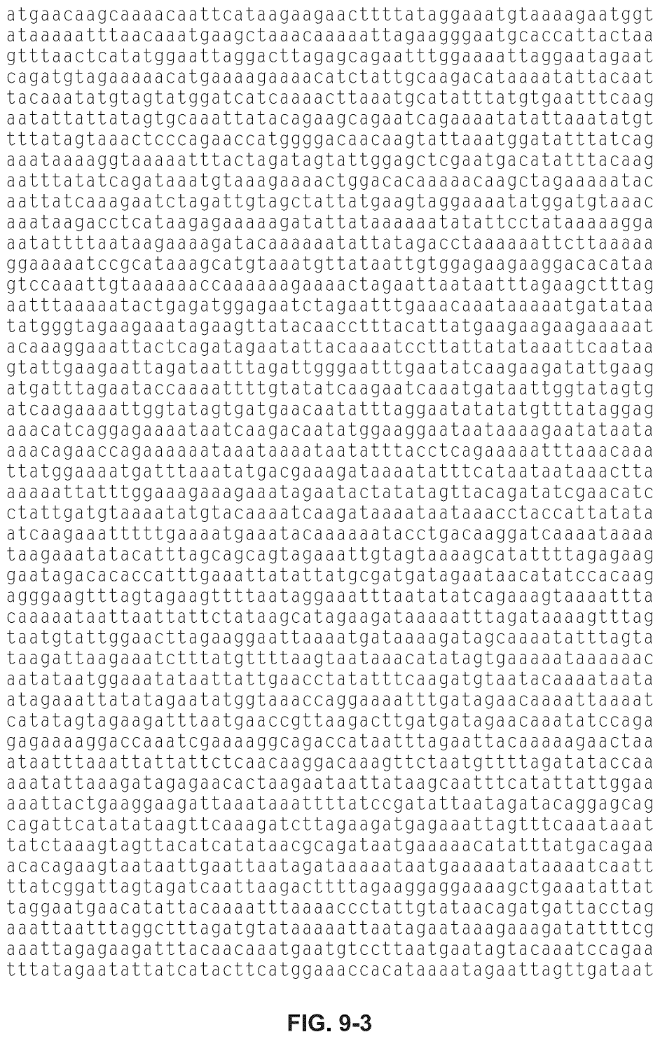

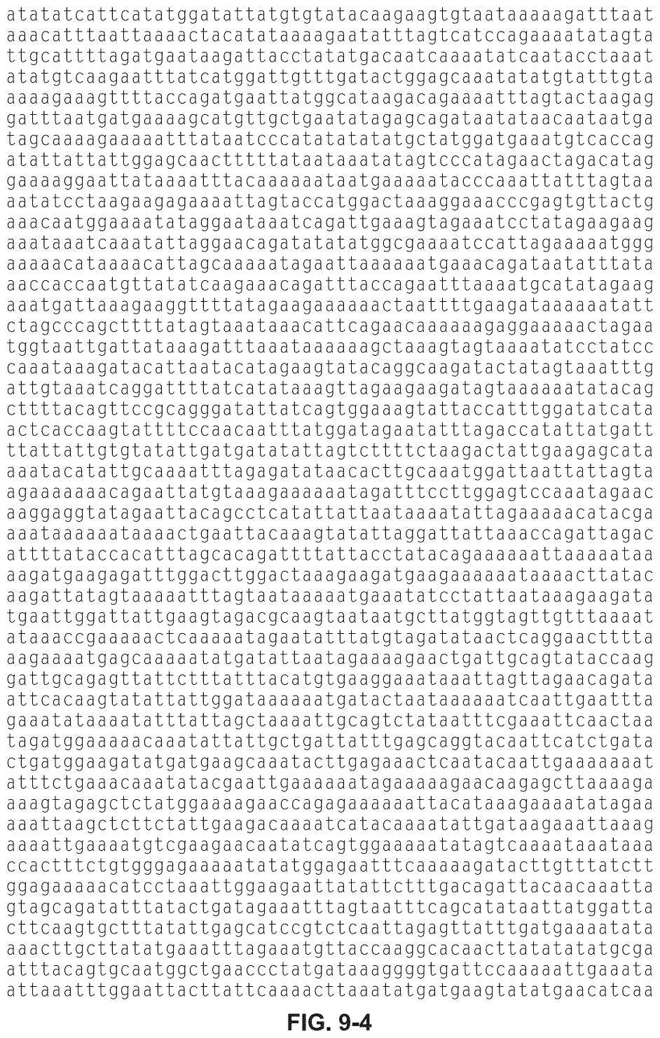

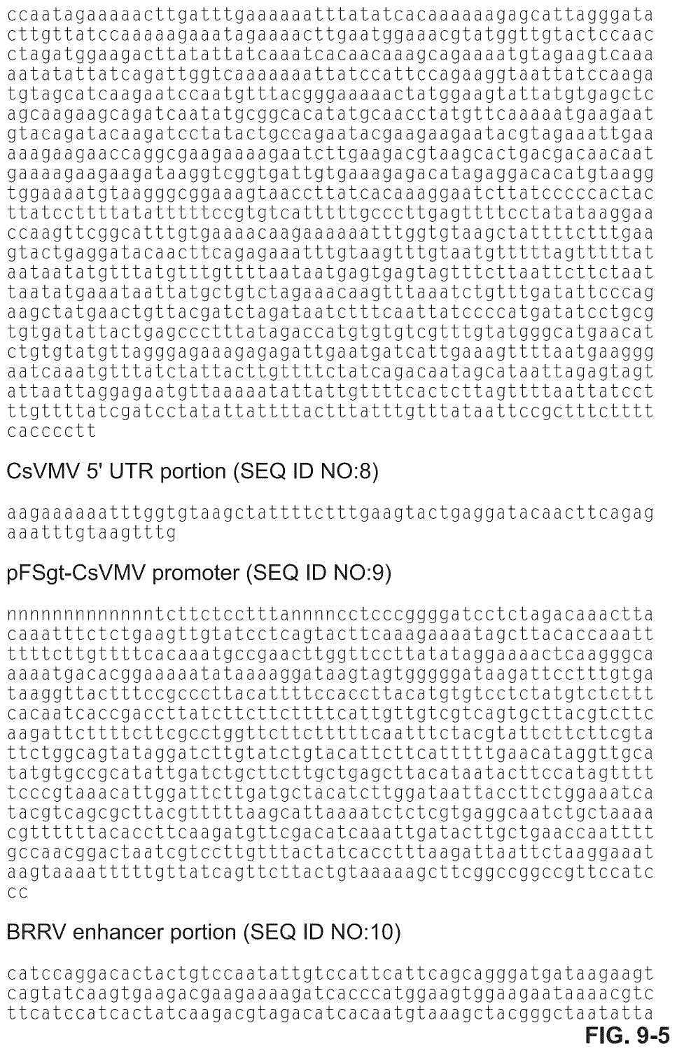

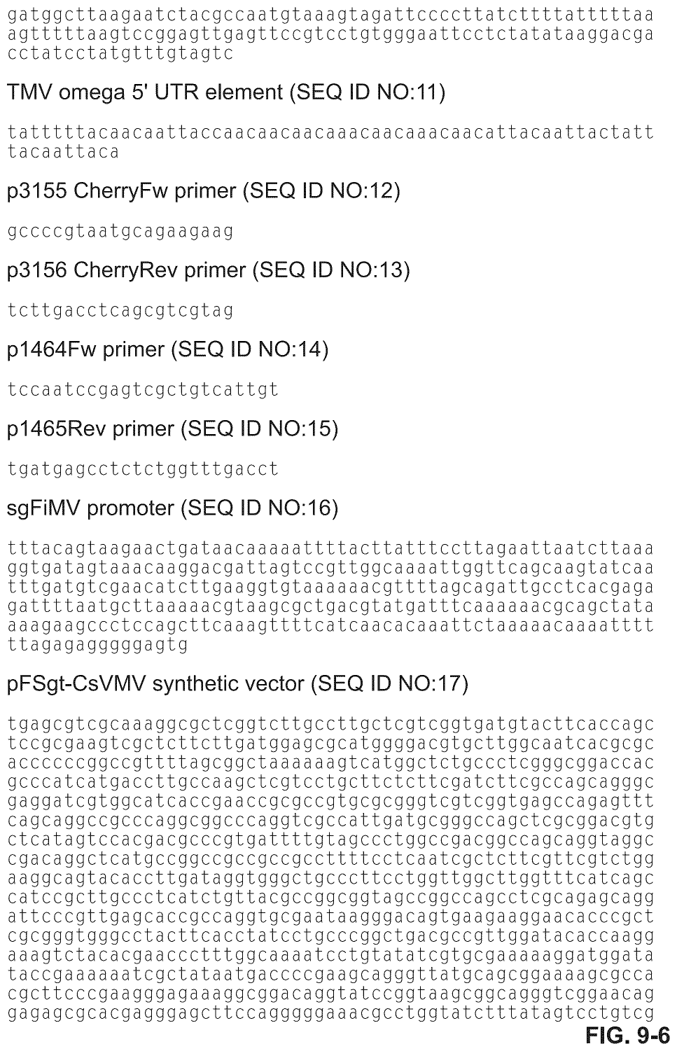

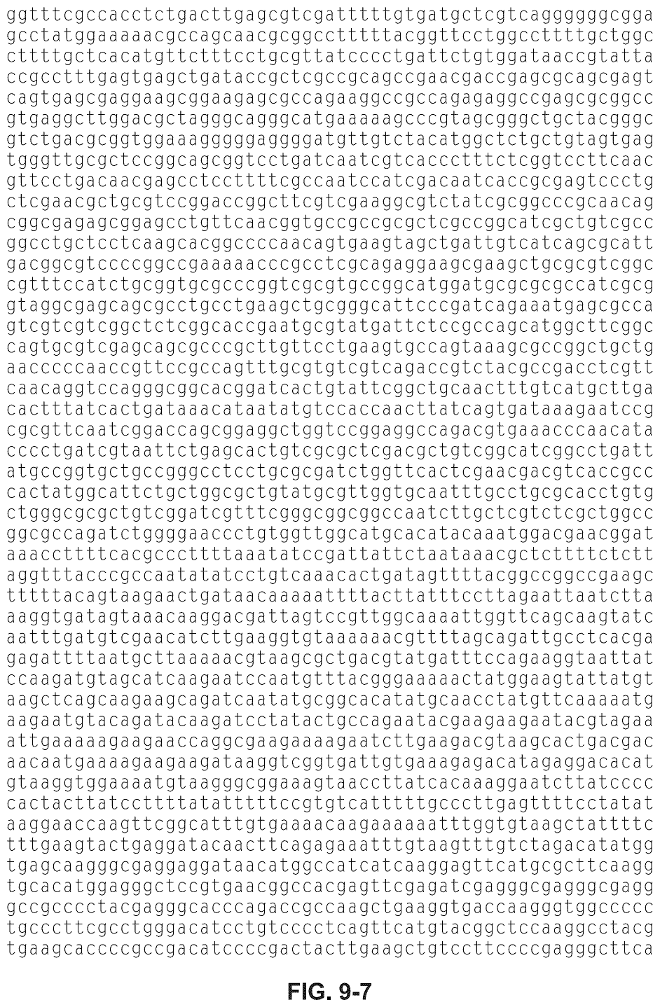

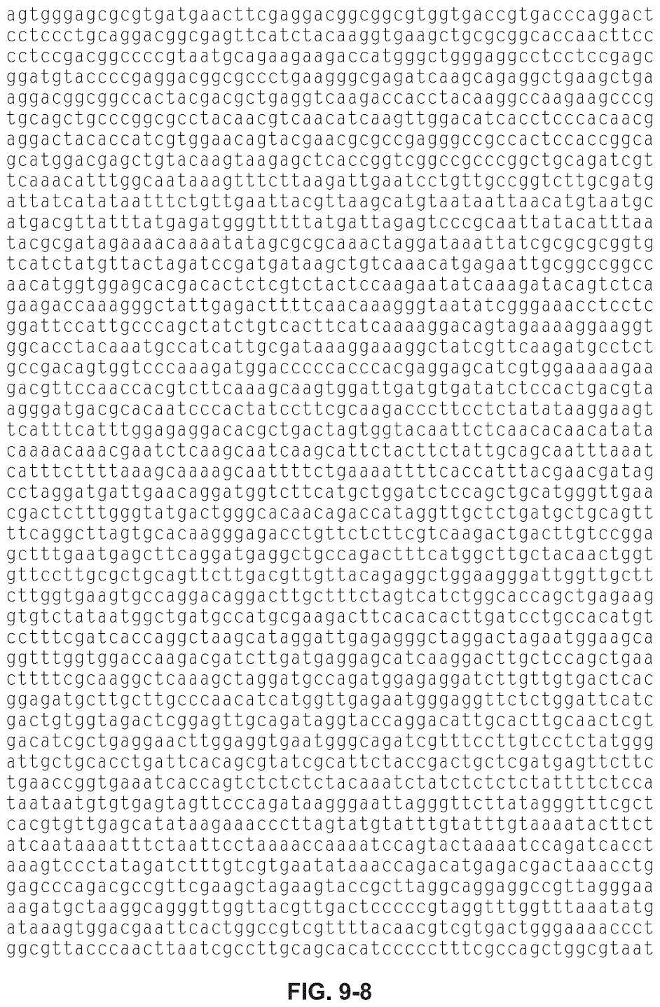

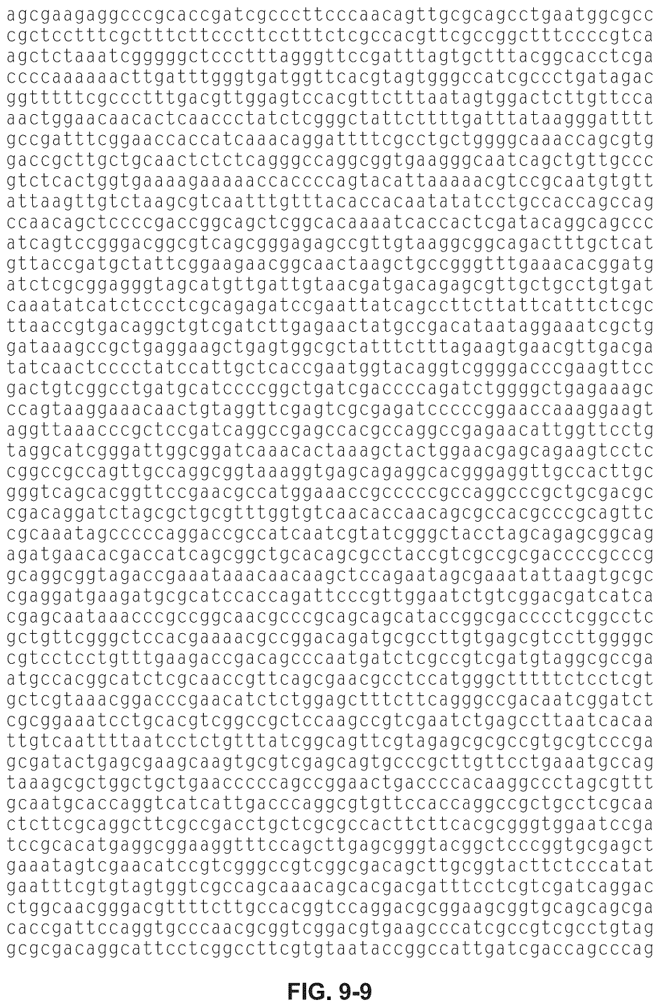

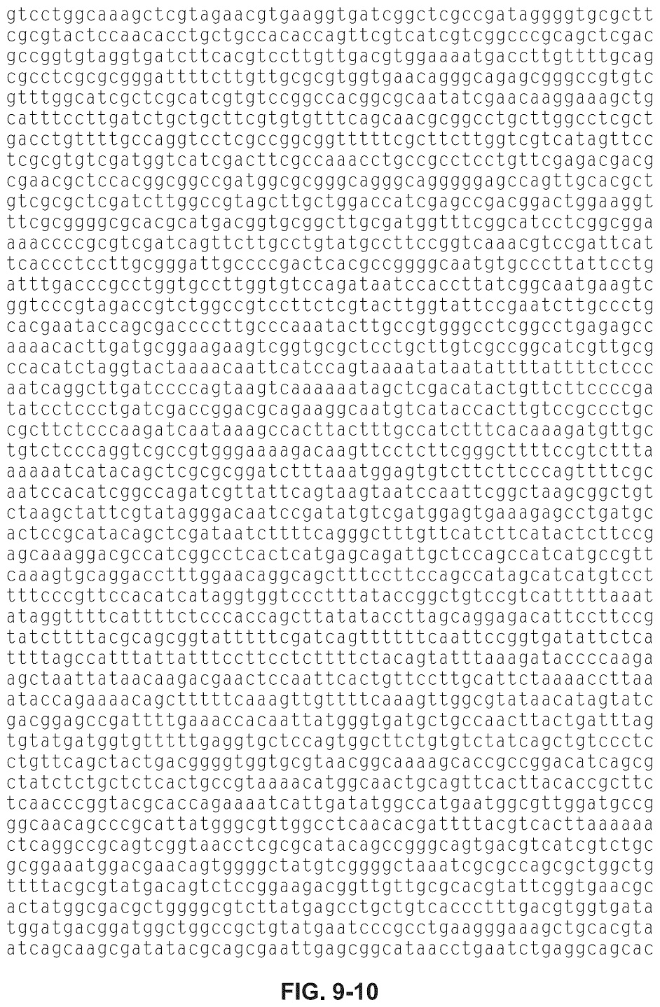

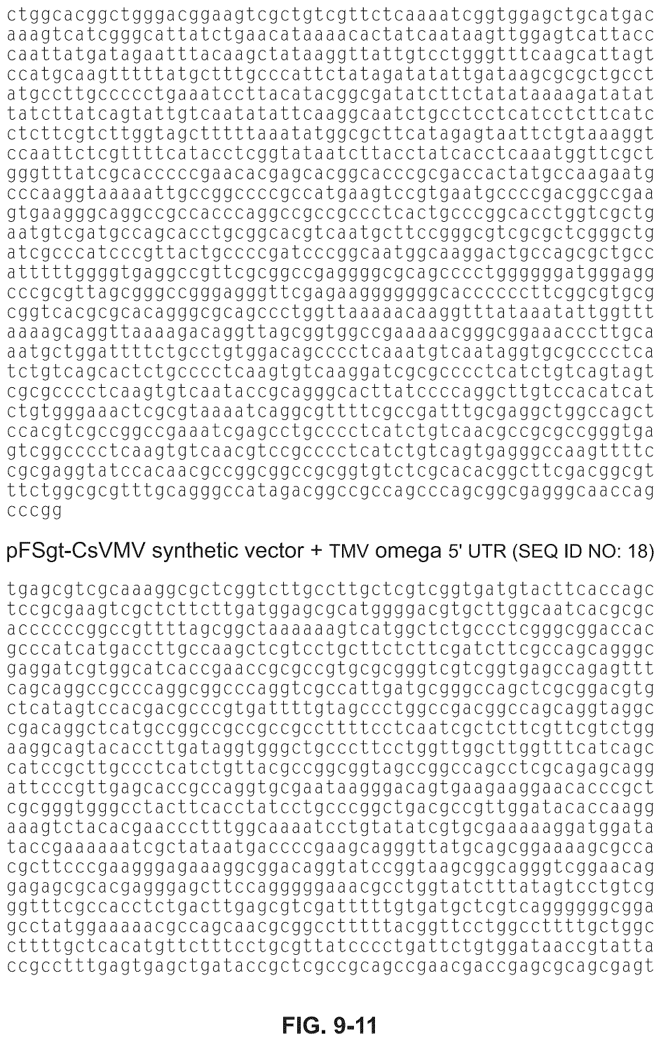

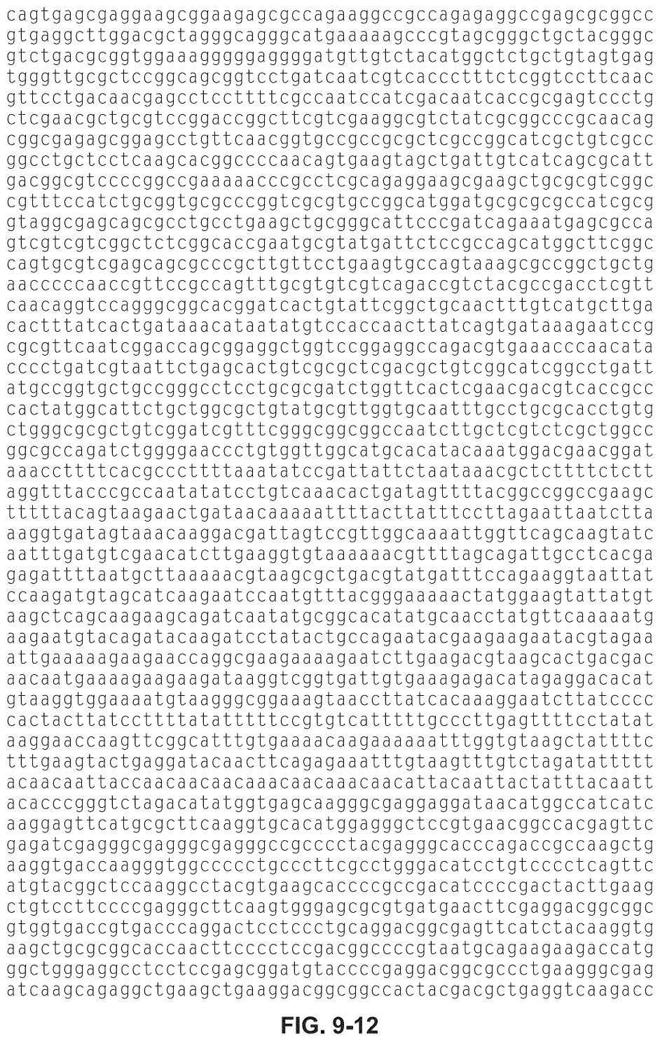

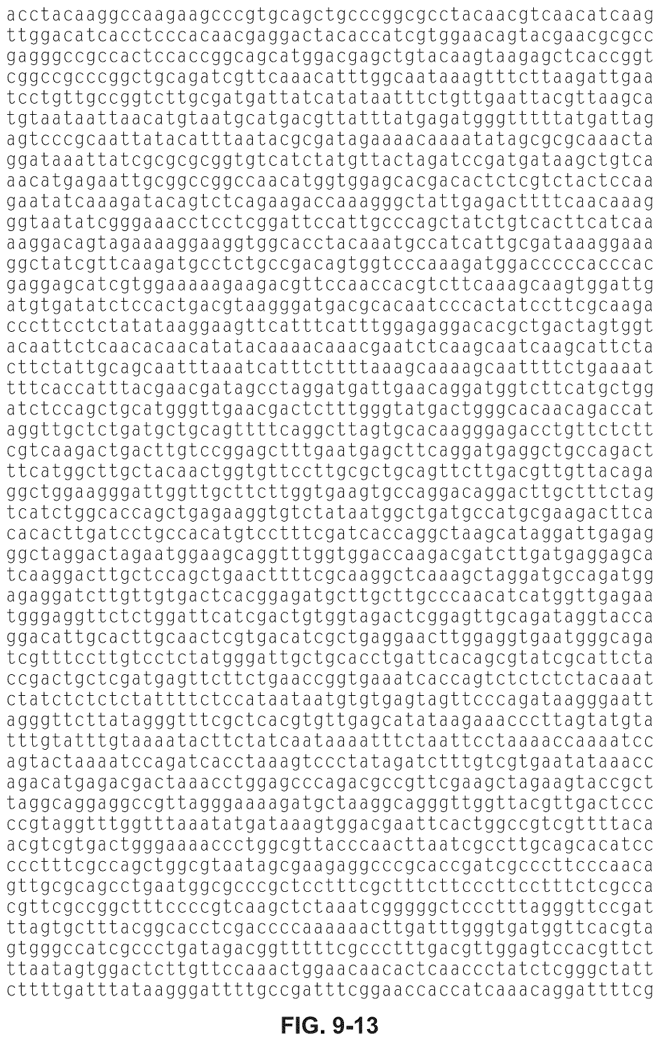

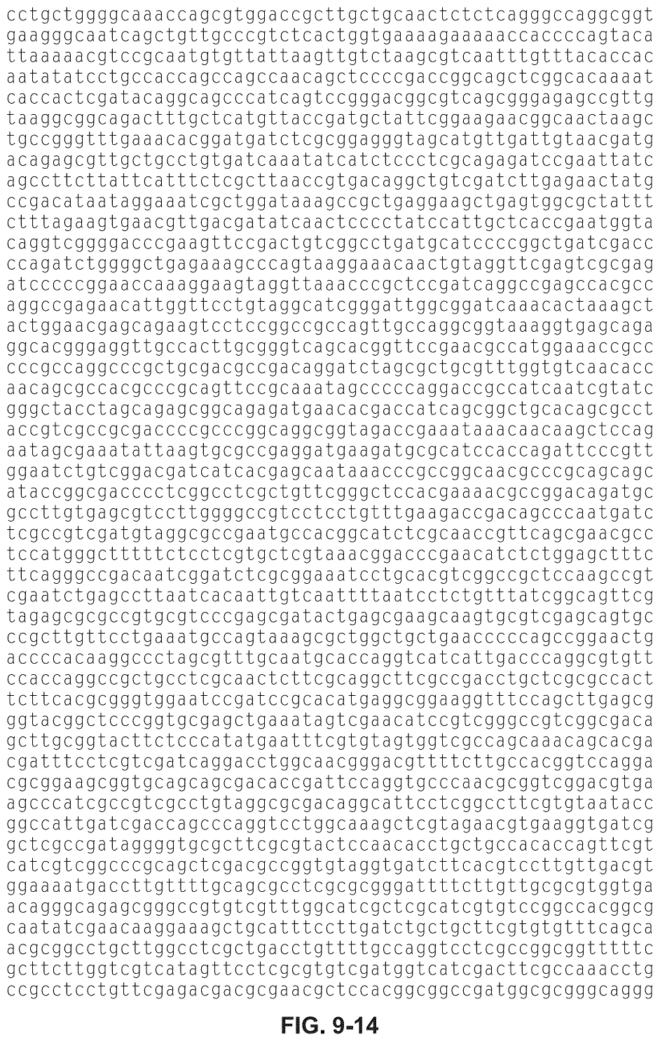

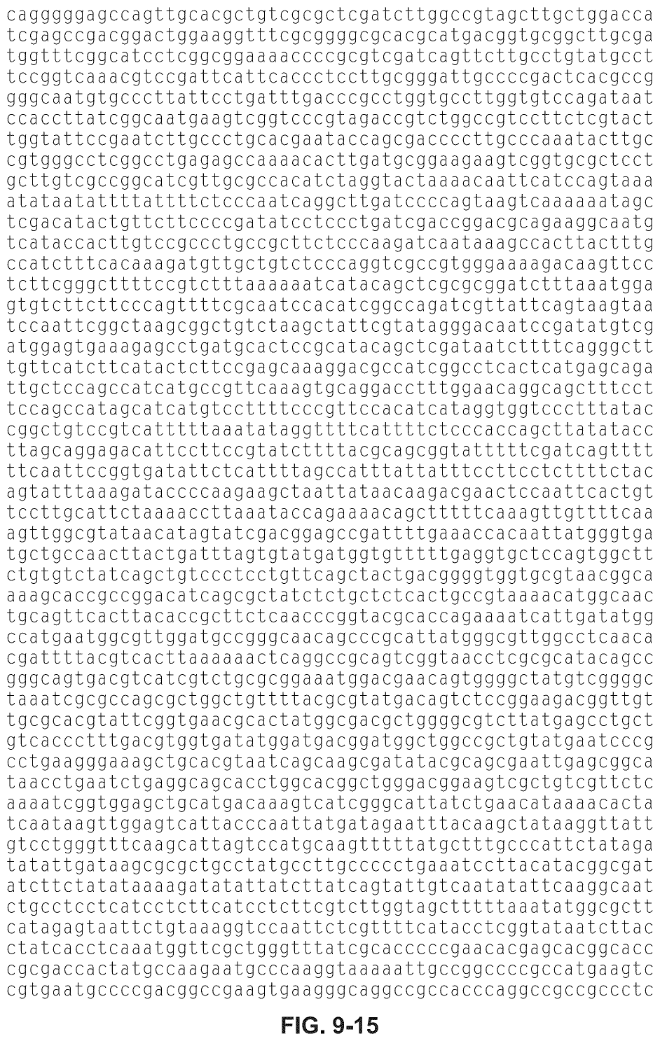

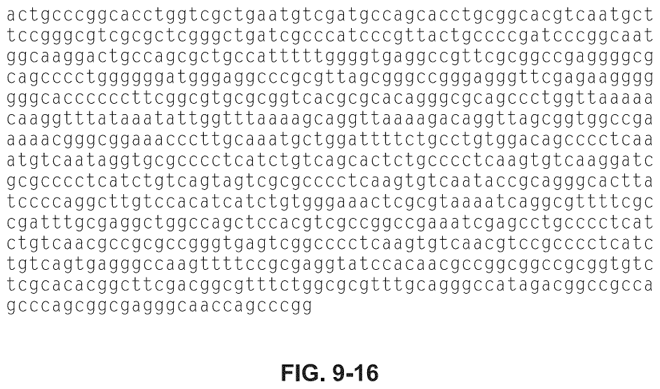

To indicate the successful transformation and the functionality of the promoter in plant tissues, the expression of mCherry was visualized in transformed explants. Specifically, florescent emission (600 nm) was visualized by Olympus SZX16 fluorescent microscope (captured by Olympus DP72 camera, and cellSense acquisition software). FIG. 8 shows images of regenerated Eucalyptus explants, with florescence in callus tissue and preliminary shoots. In order to assess the expression profile, plant tissues were sampled and assessed for expression levels by qRT-PCR and Western blot.

### Example 5: Assessment of Expression level in Plant Tissues (Transcript)

After preliminary shoots are excised and grown on elongation media in tissue culture, they are propagated and later passed to greenhouse for cultivation into mature plants. Samples from leaves, stem and root tissues of 3 mo.-old pFSgt-CsVMV-mCherry and control plants are taken, and RNA extracted using Plant/Fungi Total RNA Purification Kit (Cat. 25800, Norgen Biotek), according to manufacturer's instructions. To detect the degree of transgenic transcript expression, reverse transcription followed by real-time qPCR are used. The primer pairs for the mCherry and a ‘housekeeping’ reference transcript amplicons are listed in Table 3.

TABLE 3

| | | | |
|---|---|---|---|
| 1 | p3155 | GCCCCGTAATGCAGAAGAAG | SEQ ID NO: 12 |
| | CherryFw | | |
| 2 | p3156 | TCTTGACCTCAGCGTCGTAG | SEQ ID NO: 13 |
| | CherryRev | | |
| 3 | p1464Fw | TCCAATCCGAGTCGCTGTCA | SEQ ID NO: 14 |
| | | TTGT | |
| 4 | p1465Rev | TGATGAGCCTCTCTGGTTTG | SEQ ID NO: 15 |
| | | ACCT | |

The qRT-PCR Amplification and detection were done using an Applied Biosystems StepOnePlus™ Real-time machine.

### Example 6: Assessment of Expression Level in Plant Tissues (Protein)

For protein extraction from plant tissues, buffer composed of 50 mM Tris-HCl PH 7.5 with protease inhibitor (100X Protease Inhibitor Cocktail for plant cell and tissue extracts, DMSO solution, P9599, Merck) is used. Western blot is carried out using the E-PAGE™ 48 System from INVITROGEN, and transfer with INVITROGEN-iBlot™ Dry Blotting System, both according to manufacturer's instructions.

### Example 7: Stacking Traits With pFSgt-CsVMV and Second Constitutive Promoter

In order to provide plants with higher resistance to widely used non-selective herbicides, it is necessary to ensure sufficient expression of a combination of herbicide tolerant genes, and avoid the reduced level of expression due to use of a redundant promoter.

This can be achieved by using different promoters for each nucleic acid whose expression is desired. Agro transformation in Eucalyptus is used to introduce two coding sequences: herbicide resistance 1 and herbicide resistance 2. Specifically, CaMV 35S expressing herbicide resistance 1 and pFSgt-CsVMV expressing herbicide resistance 2 are each separately cloned into a pBI121 binary vector (Clontech), transformed into separate plants using Agro transformation as described, and the derived tolerant plants are crossed to yield a double-transgenic progeny plant.

### Example 8: Editing pFSgt-CsVMV Into the Genome of a Plant

To induce high expression of an endogenous target transcribed element (such as a coding sequence or a functional RNA), CRISPR genome editing is used to introduce the pFSgt-CsVMV sequence in proximity to the genomic location of the endogenous target sequence to be expressed via Site Directed Integration (SDI).

For this aim, a ‘strong’ promoter (e.g., CaMV 35S or pFSgt-CsVMV) is used to drive expression of Cas9 or Cas12a (AKA, Cas expression cassette). An (RNA-pol-III) U6 promoter is used to drive the expression of a gRNA targeting a genomic target sequence, proximal to the target transcribed element. The U6-gRNA is provided from the same or from a separate plasmid as the Cas expression cassette. The guideRNA sequence is compatible with the Cas9 or Cas12a, as provided in the art.

The sequence of pFSgt-CsVMV as described herein is provided on a donor-RNA template (either in a circular or linear DNA form) to replace or include the pFSgt-CsVMV in the target site. For functionality, the pFSgt-CsVMV sequence is operably incorporated in the ′5 to ′3 orientation before the ′5 end of the endogenous target transcribed element to be expressed. Further stages of selection and propagation of the edited cells into mature plants are performed as known in the art.

1. A nucleic acid sequence, comprising:
a first isolated nucleic acid portion having at least 80% sequence identity to the Figworts mosaic Virus (FMV, FiMV) ‘sub-genomic’ transcript promoter sequence portion of SEQ ID NO: 5, wherein said first nucleic acid portion lacks a promoter-TATA-box sequence; and
(ii) a second isolated nucleic acid portion comprising a nucleic acid having at least 80% sequence identity to the Casava Vein Mosaic Virus (CsVMV) sequence of SEQ ID NO: 6, wherein said second nucleic acid portion includes a promoter-TATA-box sequence.

2. The nucleic acid sequence of claim 1, wherein said first nucleic acid portion sequence has at least 90% sequence identity to SEQ ID NO: 5.

3. The nucleic acid sequence of claim 1, wherein said second nucleic acid portion sequence has at least 90% sequence identity to SEQ ID NO: 6.

4. The nucleic acid sequence of claim 2, wherein said second nucleic acid portion sequence has at least 90% sequence identity to SEQ ID NO: 6.

5. The nucleic acid sequence of claim 1, having at least 90% sequence identity to SEQ ID NO:1.

6. The nucleic acid sequence of claim 1, having at least 95% sequence identity to SEQ ID NO:1.

7. The nucleic acid sequence of claim 1, having at least 99% sequence identity to SEQ ID NO:1

8. The nucleic acid sequence of claim 1, having the nucleic acid sequence of SEQ ID NO: 1 OR SEQ ID NO: 9.

9. A construct comprising the nucleic acid sequence of claim 1.

10. The construct of claim 9, wherein said nucleic acid sequence is operably linked to (i) a nucleic acid sequence encoding a protein or (ii) a nucleic acid sequence that is transcribed to yield a functional RNA.

11. A plant cell comprising the nucleic acid sequence of claim 1.

12. A plant cell comprising the construct of claim 9.

13. A plant comprising the plant cell of claim 11.

14. The plant of claim 13, wherein the plant is a dicot plant.

15. The plant of claim 13, wherein the plant is a monocot plant.

16. A method of expressing a transcribed element in a plant, comprising a step of introducing the nucleic acid sequence of claim 1 into a plant cell.

17. The method of claim 16, wherein the nucleic acid sequence of claim 1, is introduced into a plant cell genome by gene editing methods selected from TALEN or CRISPR.

18. The method of claim 17, wherein the nucleic acid sequence of claim 1, is introduced into a plant cell genome by CRISPR Site Directed Integration (SDI).

19. The method of claim 16, wherein the plant is selected from Eucalyptus subtypes.

20. The method of claim 16, further comprising the step of selecting plant or plant cells having a desired phenotype.
